SEQUENCE LISTING (2 sequences)

SEQ ID NO: 1 - DNA, 25 bp, Artificial Sequence
  16s rDNA universal forward primer
gcggatcctctagactgcagtgcca

SEQ ID NO: 2 - DNA, 19 bp, Artificial Sequence
  16s rDNA universial reverse primer
ggttaccttgttacgactt

(19) United States
(12) Patent Application Publication
(10) Pub. No.: US 2019/0211302 A1
(43) Pub. Date: Jul. 11, 2019

(54) SYSTEMS AND METHODS OF CARBON FIXATION USING SOLVENTOGENIC CLOSTRIDIUM BEIJERINCKII

(71) Applicant: North Carolina State University, Raleigh, NC (US)

(72) Inventors: JOSE M. BRUNO-BARCENA, Raleigh, NC (US); WALTER J. SANDOVAL ESPINOLA, Raleigh, NC (US); MARI S. CHINN, Raleigh, NC (US)

(21) Appl. No.: 16/323,107

(22) PCT Filed: Aug. 4, 2017
(86) PCT No.: PCT/US2017/045571

§ 371 Date: Feb. 4, 2019

Related U.S. Application Data
(60) Provisional application No. 62/371,562, filed on Aug. 5, 2016.

(51) Int. Cl. C12N 1/20; C12P 7/16; C01B 3/00; C12M 1/107; C12P 7/28; C12P 7/06

(52) U.S. Cl. CPC C12N 1/20; C12P 7/16; C12P 7/06; C12M 21/04; C12P 7/28; C01B 3/00

(57) ABSTRACT

Described herein are systems and methods of fixing inorganic carbon using an amount of Clostridium beijerinckii.

### CROSS-REFERENCE TO RELATED APPLICATIONS

This application claims the benefit of and priority to co-pending U.S. Provisional Patent Application No. 62/371,562, filed on Aug. 5, 2016, entitled “SYSTEMS AND METHODS OF CARBON FIXATION USING SOLVENTOGENIC CLOSTRIDIUM BEIJERINCKII,” the contents of which is incorporated by reference herein in its entirety.

### STATEMENT REGARDING FEDERALLY SPONSORED RESEARCH OR DEVELOPMENT

This invention was made with government support under grant number 2011-10006-30363 awarded by USDA/NIFA. The government has certain rights to this invention.

### SEQUENCE LISTING

This application contains a sequence listing filed in electronic form as an ASCII.txt file entitled 221404-2120_ST25.txt, created on Aug. 4, 2017. The content of the sequence listing is incorporated herein in its entirety.

### BACKGROUND

Efforts to combat rising CO₂ levels by generating alternatives to fossil fuels, have included capturing greenhouse gases (GHG) into bioenergy molecules. Accordingly, research into second generation biofuels, such as butanol, has traditionally involved the heterotrophic biotransformation of alternative sugars by natural butanol-producers (e.g. solventogenic Clostridium beijerinckii), or more recently, the exploration of autotrophic species using synthetic biology techniques. However, currently there are no demonstrations of successful carbon fixation from atmospheric carbon sources using solventogenic Clostridium beijerinckii.

### SUMMARY

Described herein are systems that can contain a fermentation vessel, wherein the fermentation vessel can be configured to receive an inorganic carbon source; and a culture of solventogenic Clostridium beijerinckii (C. beijerinckii), wherein the culture of solventogenic C. beijerinckii can be contained within the fermentation vessel. The culture of solventogenic C. beijerinckii can be a mixotrophic culture of solventogenic Clostridium beijerinckii. The culture of solventogenic C. beijerinckii can be a high density culture of solventogenic C. beijerinckii. The inorganic carbon source can be a greenhouse gas. The inorganic carbon and electron source can be syngas. The carbon source can be CO₂ and electron source can be H₂. The inorganic carbon and electron source can be up to 20% (v/v) CO. The inorganic carbon source can be up to 20% (v/v) CO₂. The electron source can be up to 8% (v/v) H₂.

Also described herein are methods of fixing inorganic carbon that can include at least the step of fermenting a carbon source using a culture of solventogenic Clostridium beijerinckii (C. beijerinckii). The culture of solventogenic C. beijerinckii can be a mixotrophic culture of solventogenic C. beijerinckii. The culture of solventogenic C. beijerinckii can be a high density culture of solventogenic C. beijerinckii. the inorganic carbon and electron source can be a greenhouse gas. The carbon and electron source can be syngas. The carbon and electron source can be up to 20% (v/v) CO. the carbon source can be up to 20% (v/v) CO₂. The electron source can be up to 8% (v/v) H₂ The carbon source can be at least 5% (v/v) CO₂ and electron source 2.5% (v/v) H₂. The step of fermentation can be carried out at about 37° C. The carbon source can be syngas that can be about 9%, about 32%, about 63%, or 100%.

### BRIEF DESCRIPTION OF THE DRAWINGS

Further aspects of the present disclosure will be readily appreciated upon review of the detailed description of its various embodiments, described below, when taken in conjunction with the accompanying drawings.

FIGS. 1A-1D shows graphs that can demonstrate the results of direct monitoring of hydrogen and carbon dioxide evolution in the gas-phase during fed-batch fermentations by C. beijerinckii. H₂ and CO₂ evolution from three independent experiments performed in a Biostat B+ reactors using defined medium containing about 6% (w/v) sucrose as limiting carbon and energy source with an initial and final volumes of about 1000 mL and about 1400 mL, respectively. Feeds (about 400 mL) contained about 80 g sucrose fed at about 0.08 mL/h, to reach a final concentration of about 100 g/L (w/v) along with: Red line: only sugar was added; Black line: fresh whole medium and; Blue line: 2× trace components. Yellow boxes show detail of the H₂ and CO₂ oscillation, which are shown zoomed in FIGS. 1B and 1D respectively. Fermentations were controlled at about 250 rpm, about 37° C. and pH about 6.5 and constantly sparged (about 12.48 L/h) with nitrogen gas was achieved using mass flow controllers. Output gas-phase composition was continuously monitored and recorded using two analyzers: An EasyLine continuous analyzer, model EL3020 (ABB, Germany) and a Pfeiffer OmniStar quadrupole mass spectrometer.

FIGS. 2A-2C show cartoons (FIGS. 2A and 2B) and a table (FIG. 2C) show Wood-Ljungdahl (WL) and reverse pyruvate: ferredoxin oxidoreductase/pyruvate-formate lyase (rPFOR/Pfl) pathways scheme in C. beijerinckii and chromosome localization of corresponding genes. FIG. 2A) C-1 assimilation genes found in C. beijerinckii and C. ljungdahlii (WL pathway), mapped in their respective chromosomes. FIG. 2B) Presumed WL and rPFOR/Pfl scheme pathways in C. beijerinckii. Red arrows indicate reactions predicted to be catalyzed by the CO dehydrogenase/Acetyl-CoA synthase complex, or bifunctional CODH/ACS with a question mark (acetyl-CoA is not coded in C. beijerinckii genome, but its CODH has metal centers found in bifunctional enzymes, hence the question mark in the figure). Gray and blue arrows indicate reactions belonging to the methyl branch of the WL, and rPFOR/Pfl pathways, respectively. Black and yellow arrows indicate CO₂ and H₂ assimilation (and evolving) reactions. FIG. 2C) Genes and their names associated to C-1 assimilation in C. beijerinckii.

FIG. 3 shows a time-course transcription profile of C-1 assimilation and energy conservation genes in C. beijerinckii. Time-course expression profiles of Wood-Ljungdahl and pyruvate: ferredoxin oxidoreductase/pyruvate-formate lyase (rPFOR/Pfl) predicted genes, along with genes related to energy conservation in C. beijerinckii. The FPKM (fragments per kilobase per million) were calculated from publicly available RNA-seq data²⁷. Lines represent CO₂ and H₂ evolution in the gas-phase of C. beijerinckii growing in defined medium²¹, 37° C., 250 rpm, and constantly sparged with pure nitrogen (12.48 L/h).

FIGS. 4A-4D show graphs demonstrating kinetic, yield parameters and carbon and energy balances of steady-state cells (D=0.135 h⁻¹) cultured mixotrophically on presence of synthesis gas (CO, CO₂ and H₂). (FIG. 4A) Each tested gas mixture was constantly sparged at 12.48 L/h. Steady-state values of gas generation (H₂ and CO₂) and synthesis gas utilization were obtained after subtracting the net values of H₂ and CO₂ generated under sparged nitrogen from the values of output gas phase for each condition. Zero value indicates input=output. Negative values indicate more gases being produced than input. Positive values indicate the steady-state amounts continuously assimilated. (FIG. 4B) Sucrose and fructose consumed in steady states under the different tested gas phase condition. (FIG. 4C) Apparent yield (C-mol product/C-mol carbon source utilized) and (FIG. 4D) carbon and energy balance, calculated as

$$\sum ( \frac{Yp \cdot γ p}{γ s} ) + \frac{Yx \cdot γ x}{γ s} = 1 ,$$

where Yp and Yx represent C-mol ratios on figure (FIG. 4C); γ represents electrons available for each fermentation product⁵⁶. Synthesis gas mixture was balanced with nitrogen (100% synthesis gas contains 20% CO, 20% CO₂, 10% H₂ and 50% N₂). The results presented here were obtained from three biological replicates and the represented means are values at steady-state from at least three samples extracted at different retention time intervals. Significance at 0.05 refers to comparisons between whole columns.

FIG. 5 NMR spectra of C-13 labeled products. Cultures of C. beijerinckii SA-1 growing mixotrophically and exposed to high synthesis gas concentration (60% synthesis gas 40% N₂, where 100% synthesis gas contains 20% CO, 20% CO₂, 10% H₂ and 50% N₂) were collected at steady state (D=0.135 h-1) and grown in batch during 48 h in presence or absence of ¹³C labeled CO₂. NMR ¹³C spectra of culture media from: FIG. 5A) Control, C. beijerinckii grown in the presence of 100% [v/v] CO₂ in the headspace; FIG. 5B) C. beijerinckii grown in the presence of 100% [v/v] ¹³C labeled CO₂ tracer in the headspace; FIG. 5C) Culture media from control cultures supplemented with standards butyric (9 μg) and acetic acid (7.68 μg). All samples contained as internal standard 0.081 μg of C-13 methanol.

FIGS. 6A-6C show tables demonstrating the results from a transcription analysis of C. beijerinckii C-1 assimilation and energy conservation pathways. RNA was extracted from cultures growing in chemostat (D=0.135 h-1) sparged (12.48 L/h) either with N₂, low (9%), or high (60%) concentrations of synthesis gas. Cells were cultivated, in defined media21, 250 rpm, 37° C. and pH 6.5. (100% synthesis gas contains 20% CO, 20% CO2, 10% H₂ and 50% N₂). Statistical significance: 0.05>p>0.005=*; 0.005>p>0.0005=**; 0.0005>p>0.00005***p<01×10⁻⁵=*****.

FIGS. 7A-7F show graphs that can demonstrate transient responses to nitrite pulses by C. beijerinckii growing in chemostat (D=0.135 h⁻¹). Experiments were performed on defined medium containing 3% sucrose and 1.5% fructose (w/v) and sparged with 60% (v/v) synthesis gas balanced with nitrogen (100% synthesis gas (syngas) contains 20% CO, 20% CO₂, 10% H₂ and 50% N₂) at 37° C. The additions of sodium nitrite are indicated with vertical dashed lines to reach final concentrations as follow; 3.1 mM (FIG. 7A), 6.2 mM (FIG. 7B) and 12.4 mM (FIG. 7C). The CO and H₂ data shown were obtained by monitoring, in real time, with an EasyLine continuous gas analyzer, model EL3020 (ABB, Germany). Nitrite concentrations higher than 24 mM proved toxic and led to washout. Steady-state values were re-established prior to testing each nitrite concentration. Correlations of NO₂ added with FIG. 7D) H₂ consumed, FIG. 7E) amount of CO consumption displaced, and FIG. 7F) biomass increase, were calculated from the slopes after fitting the data to linear regressions.

FIG. 8 shows a logic model of carbon-electron flow in C. beijerinckii grown mixotrophically. The data suggest that in the presence of CO and CO₂, there are three possible paths for carbon capture: 1) CO₂ to carbonate through carbonic anhydrase, or 2) CO oxidation to generate CO₂+H, if the CO₂ in the gas-phase is <5% (v/v); or finally, 3) the Wood-Ljungdahl pathway, if CO₂>5%. Simultaneously, supplied sugars proceed to glycolysis. In the absence of an electron bottleneck, ABE-fermentation utilizes all the sugar-derived carbon and electrons. Otherwise, and if no external electron sink is provided, fermentation stops. In the presence of an external electron sink (such as CO/CO₂), #2 or #3 take place. If #3 takes place, the extra acetyl-CoA generated, along with the still-running ABE-pathway, leads to up to 17 and 27% more carbon and carbon-energy recovered, respectively.

FIG. 9 shows a graph demonstrating the mean growth curve of multiple fed-batch fermentations of C. beijerinckii. Two slopes, at early exponential (mₐ) and late exponential (m_{b}) growth phases, respectively, are shown. μₐ and μ_{b} are early and late specific growth rates, respectively.

FIGS. 10A-10H shows graphs that can demonstrate product and substrate profiles of fed-batch fermentations of C. beijerinckii. Different feed nutritional compositions were used: Circle: whole medium+sucrose; Triangle: sucrose only and Square: 2× trace components+sucrose. Experiments were performed in a defined medium (18) containing an initial amount of 6% (w/v) sucrose as limiting carbon and energy source. The final sucrose concentration (feed+initial medium) was 100 g/L, fed at 0.08 mL/h. Temperature was 37° C. and pH was controlled (about 6.5). initial and final volumes were 1 and 1.4 L, respectively. Nitrogen gas was flowed filtered-sterilized at 12.48 l/h throughout each experiment. FIGS. 10A and 10B: Butanol; FIGS. 10C and 10D: Acetone; FIGS. 10E and 10F: Ethanol and; FIGS. 10G and 10H: residual sucrose. FIGS. 10B, 10D, 10F, and 10H show the mean values and 95% confidence limits of butanol, acetone, ethanol and sugar, respectively. The vertical lines in FIG. 10G represent the time at which each feed was started: solid: whole medium+sucrose; dashed: sugar only and; dots: 2×trace components+sucrose. The solid vertical line in FIG. 10H represents the mean time at which feed was started. Error bars indicate SD.

FIG. 11 shows a table demonstrating the final kinetic and yield parameter. Values were obtained after different fed-batch fermentations of C. beijerinckii at 37° C., pH 6.5 and feed at 0.08 mL/h, in defined media.

FIGS. 12A-12C show graphs that can demonstrate CO₂ and H₂ consumption, carbon recovery and energy balance. FIG. 12A shows steady-state values Steady-state values (D=0.135 h⁻¹) of CO2 and H2 utilization calculated as absolute values of amount of H₂ and CO₂ produced by the cells under nitrogen conditions plus exogenous gases minus output. Positive values indicate the amount that the cells continuously assimilate, at a flow of 12.48 L/h. (FIG. 12B) Yield C-mol ratio and (FIG. 12C) carbon and energy balance, calculated as

$$\sum ( \frac{Yp , γ p}{γ s} ) + \frac{Yx , γ x}{γ z} = 1 ,$$

where Yp and Yx represent C-mol ratios of each product and biomass; γ represent electrons available. The results presented here were obtained from three biological replicates and the represented means are values at steady-state conditions from at least three samples extracted at different retention time intervals. Significance at 0.05 refers to comparisons between whole columns.

FIG. 13 shows a table demonstrating carbon and carbon and energy balances of chemostat (D-0.135 h⁻¹) cultures of C. beijerinckii SA-1 continuously sparged with CO₂ and H₂ (F=12.48 L/h).

FIG. 14 shows a table demonstrating the experimental percentage of measured input syngas. Inputs were balanced with N2, and corresponding values of its components as percentage of volume and millimols per hour. Flow: 12.48 L/h. Syngas 100% contains 20% CO, 20% CO₂, and 10% H₂.

FIG. 15 shows a table demonstrating carbon and energy balances of chemostat (D=0.135 h⁻¹) cultures of C. beijerinckii SA-1 continuously sparged with different concentrations of synthesis gas (F=12.48 L/h).

FIGS. 16A-16C show graphs demonstrating syngas (100%) consumption, carbon recovery and carbon and energy balance. (FIG. 16A) shows steady-state values (D=0.135 h⁻¹) of syngas utilization (CO, CO₂ and H₂) calculated as absolute values of amount of H₂ and CO₂ produced by the cells under nitrogen conditions plus exogenous gasses minus output. Flow=12.48 L/h. FIG. 16B shows a graph demonstrating yield C-mol ratio. FIG. 16C shows a graph demonstrating carbon and energy balance, calculated as

$$\sum ( \frac{{Y}_{P} \cdot {y}_{P}}{ys} ) + \frac{Yx , yx}{ys} = 1 ,$$

where Yp and Yx represent C-mol ratios of each product and biomass; γ represent electrons available. The results presented here were obtained from three biological replicates and the represented means are values steady-state conditions from at least three samples extracted at different retention time intervals. Significance at 0.05 refers to comparisons between whole columns.

FIGS. 17A-17K show graphs and plots demonstrating time-point geneome-wide expression profile and comparison of C. beijerinckii NCIMB 8052. FIGS. 17A-17F show graphs demonstrating time-point genome-wide expression levels (log₂ of fragment per kilo base per million—FPKM—), minus housekeeping genes (HKG) and those whose expression were below log₂=2, in C. beijerinckii NCIMB 8052, highlighting Wood-Ljungdahl (WL) pathway genes. FIGS. 17G-17K show volvano plots comparing expression levels of each time point. Alpha: 0.1; fold-change considered: 1 log₂ fold.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, organic chemistry, biochemistry, botany and the like, which are within the skill of the art. Such techniques are explained fully in the literature.

### Definitions

As used herein, As used herein, “about,” “approximately,” and the like, when used in connection with a numerical variable, can generally refers to the value of the variable and to all values of the variable that are within the experimental error (e.g., within the 95% confidence interval for the mean) or within +/−10% of the indicated value, whichever is greater.

As used herein, C₄ compounds can include any compound having 4 carbon atoms that can be in any configuration (e.g. straight chain, branched, or otherwise configured). Likewise, C₃, C₅, C₆, C₇, C₈ and so forth and so on can be any compound having 3, 5, 6, 7, 8, etc. carbon atoms present in any configuration.

As used herein, “solventogenic” can refer to organisms that can produce solvents.

As used herein, “acetogenic” can refer to organisms that can only produce acetate typically via anaerobic respiration.

As used herein, “mixotrophic” can refer to organisms that can utilize a mix of different sources of energy and carbon.

Discussion

Carbon monoxide (CO) has been used to inhibit evolving hydrogenases of the solventogenic Clostridium acetobutylicum, leading to an increase of butanol titers by the redirection of electrons from hydrogenases to solvents. The inhibition of the hydrogenases by the CO leads to increases in butanol titers but it cannot be described as inorganic carbon capture per se. (U.S. Pat. No. 4,560,658). Additionally, it was reported in the prior art the assimilation of CO into ethanol by natural acetogenic Clostridia using either pure or syntrophic cultures (e.g. U.S. Pat. Nos. 6,136,577, 8,354,257; US Pat. App. Pub. No.: 2008/0305540; International Pat. App. Pub. Nos.: WO 2013/124,401 A1, WO 2014/113,209 A1); or into butanol by genetically designed acetogenic Clostridia or by wild-type mixed cultures of acetogenic and solventogenic clostridia (syntrophic cultures) (e.g. US Pat. App. Pub. No.: 2014/234,926 A1 and U.S. Pat. No. 8,129,155). However, these methods generated low final titers of the C4-solvents in question. The efforts using only natural acetogenic Clostridia able to fix inorganic carbon cannot generate butanol or other C4 compounds. Additionally, during C4 solvent production, is desirable high biomass density, which is currently limited by the known techniques. These inefficiencies render current techniques impractical for use at industrial scale.

With the deficiencies of current methods in mind, described herein are systems and methods for fixating inorganic carbon using solventogenic C. beijerinckii. In embodiments, the systems and methods provided herein can produce C4 solvents and C4 organic acids, including butanol and butyrate, from inorganic carbon and electron sources, such as a greenhouse gasses, using solventogenic C. beijerinckii. The systems and methods provided herein can have increased yields of products, particularly butanol and butyrate, as compared to traditional fermentation processes. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

The systems and methods described herein can utilize C. beijerinckii to fix inorganic carbon. The system can include a mixotrophic pure culture of C. beijernckii capable of capturing syngas components (e.g. CO, CO₂, and H₂) and/or CO₂ and H₂ alone, into products, including but not limited to butyrate and butanol. The C. beijerinckii that can be included in the system can be capable of inorganic carbon fixation, a characteristic only attributed previously to acetogenic Clostridia. Further, C. beijerinckii can be capable of generating over the theoretical C₄ carbon recovery yields up to about 85%. Previous attempts to fix inorganic carbon in to C₄ compounds required CO₂ to be present at 100% using acetogenic organisms. In other words, in previous attempts required CO₂ to be the only carbon source. As described and demonstrated herein, this is not required by the systems and methods herein, thus making them more efficient than currently known and available systems and methods for fixing inorganic carbon. Further the systems and methods herein can allow for fixing inorganic carbon into C₄ compounds in a single step using a single solventogenic industrial organism, which is not achieved by any currently known techniques.

The system can contain a fermentation vessel, where the fermentation vessel can be configured to receive and/or contain a carbon source and/or other feed source (e.g. a sugar or alcohol) and a culture of a solventogenic Clostridium beijerinckii (C. beijerinckii). In some embodiments, the culture of solventogenic C. beijerinckii is contained within the fermentation vessel. The fermentation vessel can contain one or more inlets to allow the carbon source and/or other feed source, and/or C. beijerinckii culture enter the vessel before and/or during fermentation. The fermentation vessel can also contain one or more outlets that can be configured to allow the removal of fermentation (or harvest) product during and/or after fermentation. The fermentation vessel can be any suitable size or shape. In some embodiments, the fermentation vessel is about 2 L or more. The fermentation vessel can be configured to receive inorganic and/or organic carbon sources. In some embodiments, the inorganic carbon source can be syngas and/or CO₂ together with H₂. The fermentation can also include one or more mass flow controllers. These can be operated to modify the inlet gasses or other feed sources entering the fermentation vessel.

The fermentation vessel can be fluidically or otherwise coupled to a carbon source. The culture of solventogenic C. beijerinckii can fix inorganic carbon and simultaneously proliferate heterotrophically (also referred to herein as mixotrophic growth). The culture of solventogenic C. beijerinckii can be a high density culture of solventogenic C. beijerinckii. The electron and carbon source can be 0%, 5%, 10%, or 15 up to 20% (v/v) CO. The inorganic carbon source can be 0%, 5%, 10%, or 15 up to 20% (v/v) CO₂. The electron source can be up to 8% (v/v) H₂. In some embodiments, the inorganic electron and carbon source can be up to 20% (v/v) CO, inorganic carbon up to 20% (v/v) CO₂, and the electron source up to 8% (v/v) H₂. The inorganic carbon source can be, in some embodiments, at least 5% (v/v) CO₂ and the electron source can be 2.5% (v/v) H₂. The volume of inorganic carbon the gas-phase and under high cell density (at least OD_{600 nm} about 7) in an aqueous medium. In some embodiments, the system can be configured to continuously capture and fix inorganic carbon, such as form syngas or CO₂ and H₂.

In some embodiments, the carbon source can be syngas can be 100% syngas. 100% syngas can contain about 20% CO, about 20% CO₂, about 10% H₂ and about 50% N₂. In other embodiments, the syngas can be about 9% (low), about 32% (medium), or about 60% (high) syngas concentrations (v/v) balanced with nitrogen. In some embodiments, the amount of the CO in the syngas can range from about 10% to about 30%, the amount of CO₂ can range from about 10% to about 30%, the amount of H₂ can range from about 5% to about 15% and N₂ can range from about 25% to about 75%.

The inorganic electron and carbon sources can be the product of another fermentation or combustion process, such as a stream produced from the reforming of natural gas or from the gasification of coal or another biomass. In this way, the systems provided herein can be added onto existing manufacturing methods to capture inorganic carbon and electrons from waste from other systems and processes and produce value-added carbon compounds, such as C₄ compounds (e.g. butanol and/or butyrate). The system can be configured to receive the product from another fermentation or combustion process. In some embodiments, the fermentation vessel can be configured to receive the product from another fermentation or combustion process. In some embodiments, the fermentation can be fluidicially or otherwise coupled to an outlet from another fermentation or combustion system.

Also described herein are methods of assimilating inorganic carbon from a carbon source. The methods include at least the step of fermenting one or more carbon sources by exposing the carbon source to a culture of solventogenic Clostridium beijerinckii (C. beijerinckii) for a suitable amount of time. The fermentation mix can also include an amount of sugar, including but not limited to, sucrose and/or fructose. The amount of sugar can range from about 1% (w/v) to about 10% (w/v). The sugar can be continuously fed into the fermentation mix during the fermentation step. In some embodiments, the amount of sugar is about 6% (w/v). In some embodiments the step of fermentation can be carried out for a suitable amount of time. During the fermentation step, once the cells present reach the exponential growth stage under sparged pure nitrogen (OD600 nm is about 1), the feed flow (carbon source(s)) and harvest flow can be initiated and adjusted to a suitable dilution rate. In some embodiments, the dilution rate can be about 0.135 h⁻¹. Different gas-phase conditions, such as from pure nitrogen gas to increased syngas concentrations, can be modified during fermentation by modifying the mix ratios between syngas and nitrogen and/or other gases entering the fermentation vessel. Modification of the mix ratios can be controlled via one or more mass flow controllers that can be coupled to the fermentation vessel. The fermentation can be carried out at a temperature of about 37° C.

The method can further contain the step of purifying a carbon product, such as a C₄ compound, after or during the step of fermenting the organic and inorganic carbon sources. Suitable methods of purifying the carbon product will be appreciated by those of ordinary skill in the art in view of this disclosure. The method can produce C₄ compounds, such as butyrate and butanol. The culture of solventogenic C. beijerinckii can be a mixotrophic culture of solventogenic C. beijerinckii. The culture of solventogenic C. beijerinckii can be a high density culture of solventogenic C. beijerinckii. The electron and carbon source can be 0%, 5%, 10%, or 15 up to 20% (v/v) CO. The inorganic carbon source can be 0%, 5%, 10%, or 15 up to 20% (v/v) CO₂. The electron source can be up to 8% (v/v) H₂. In some embodiments, the inorganic electron and carbon source can be up to 20% (v/v) CO, inorganic carbon up to 20% (v/v) CO₂, and the electron source up to 8% (v/v) H₂. The inorganic carbon source can be, in some embodiments, at least 5% (v/v) CO₂ and the electron source can be 2.5% (v/v) H₂. The volume of inorganic carbon the gas-phase and under high cell density (at least OD_{600 nm} about 7) in an aqueous medium (In some embodiments, the system can be configured to continuously capture and fix inorganic carbon, such as form syngas or CO₂ and H₂.

In some embodiments, the carbon source can be syngas can be 100% syngas. 100% syngas can contain about 20% CO, about 20% CO₂, about 10% H₂ and about 50% N₂. In other embodiments, the syngas can be about 9% (low), about 32% (medium), or about 60% (high) syngas concentrations (v/v) balanced with nitrogen. In some embodiments, the amount of the CO in the syngas can range from about 10% to about 30%, the amount of CO₂ can range from about 10% to about 30%, the amount of H₂ can range from about 5% to about 15% and N₂ can range from about 25% to about 75%.

The inorganic electron and carbon source(s) can be the product of another fermentations or combustion processes, such as a stream produced from the reforming of natural gas or from the gasification of coal or another biomass. In this way, the systems provided herein can be added onto existing manufacturing methods to capture inorganic carbon and electrons from waste from other systems and processes and produce value-added carbon compounds, such as C₄ compounds (e.g. butanol or butyrate). The method thus can further include the step of obtaining a waste stream from another fermentation or combustion process, such as natural gas reformation or gasification of coal or biomass. The waste stream, more specifically compounds within the waste stream, can then be fermented by solventogenic C. beijerinckii to produce carbon-based compounds (e.g. C4 compounds such as butanol and butyrate). In this way the methods provided herein can be part of and/or applied in multi-stage fermentations where gases (e.g. CO, CO₂, and H₂) from a fermentation or combustion process can be recirculated and fermented by solventogenic C. beijerinckii to produce value-added carbon-based compounds (e.g. C4 compounds such as butanol and butyrate).

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

Introduction

Current societal efforts require solutions to address increasing greenhouse gas emissions¹. Accordingly, carbon-capture and its biotransformation into useful value-added commodities, including biofuels, has become a growing area of research. Thanks to a better understanding of pathways such as the Wood-Ljungdahl (WL), and the recently described reversed-pyruvate ferredoxin oxidoreductase (rPFOR)/pyruvate-formate-lyase-dependent (Pfl) carbon assimilation², more microorganisms can potentially be screened for presence of these pathways based via indirect physiological signals. These pathways allow the incorporation of one-carbon (C-1) molecules into Acetyl-CoA. This is a key molecule that can be subsequently bio-transformed into value-added molecules such as acetate or ethanol³⁻⁷. Currently, butanol is considered one of the ideal advanced renewable fuel due to a number of favorable properties and applications⁸⁻¹⁰. For example, it can be used unblended in unmodified car engines and is compatible with current oil infrastructure¹¹. However, only recently the assimilation of synthesis gas (containing H₂, CO and CO₂) into butanol, by natural or genetically modified microbes, has been assessed, and remains in the early stages of development^{5,6,12-14.} As a result, seeking to achieve cost-competitive butanol production, most research has focused on assessing the heterotrophic biotransformation of renewable feedstock by traditional solventogenic Clostridia. However, heterotrophic fermentations have the inherent limitation that ⅓ of carbon is lost in the form of CO₂. Interestingly, the reported data shows significant variability in apparent final product yields, pointing towards overlooked metabolic capabilities^{9,15-18}. With this in mind, this Example demonstrates results of examination of the evolving fermentation gases as physiological signals, while assessing the assimilation of synthesis gas by the natural n-butanol producer C. beijerinckii.

Results.

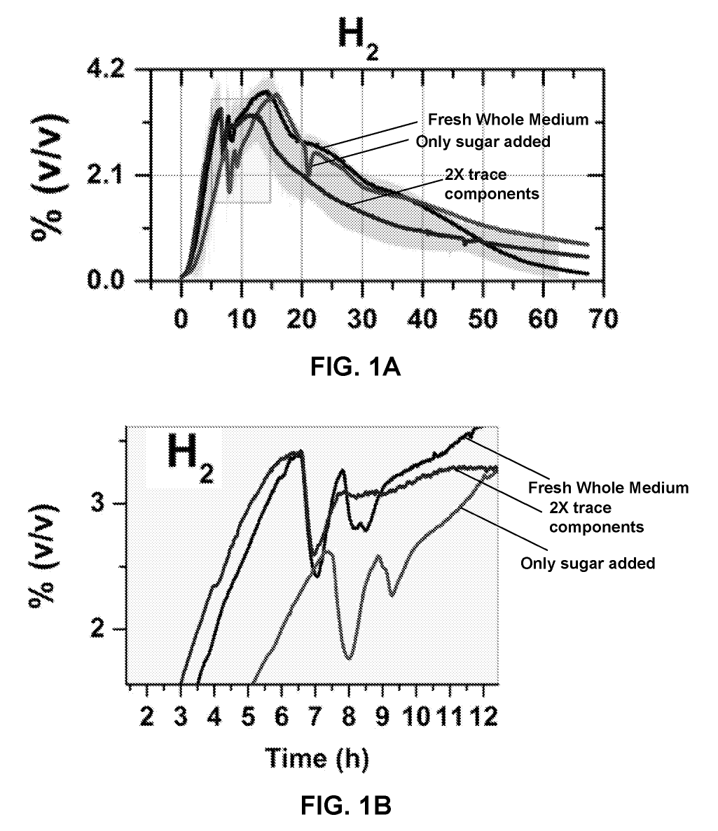

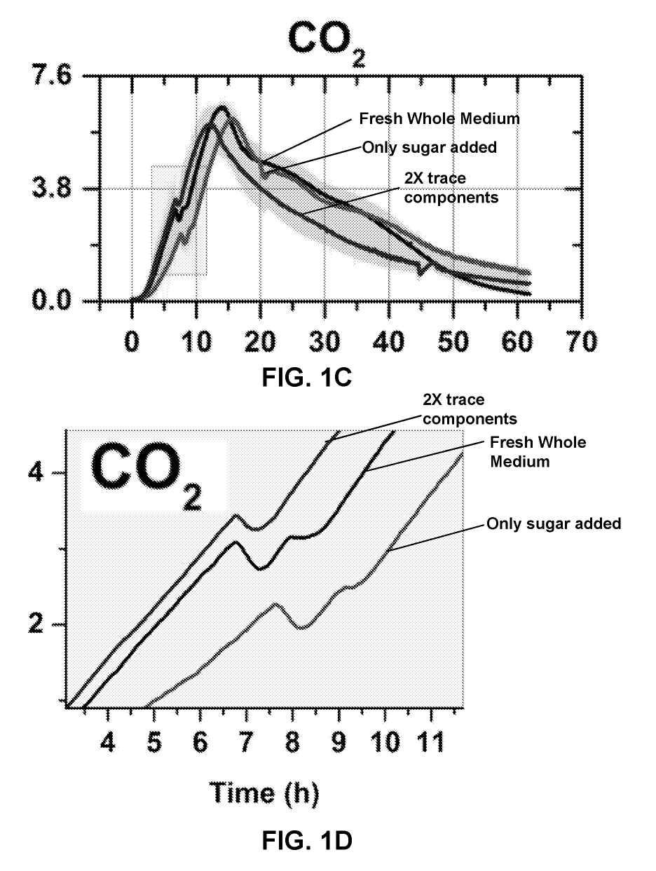

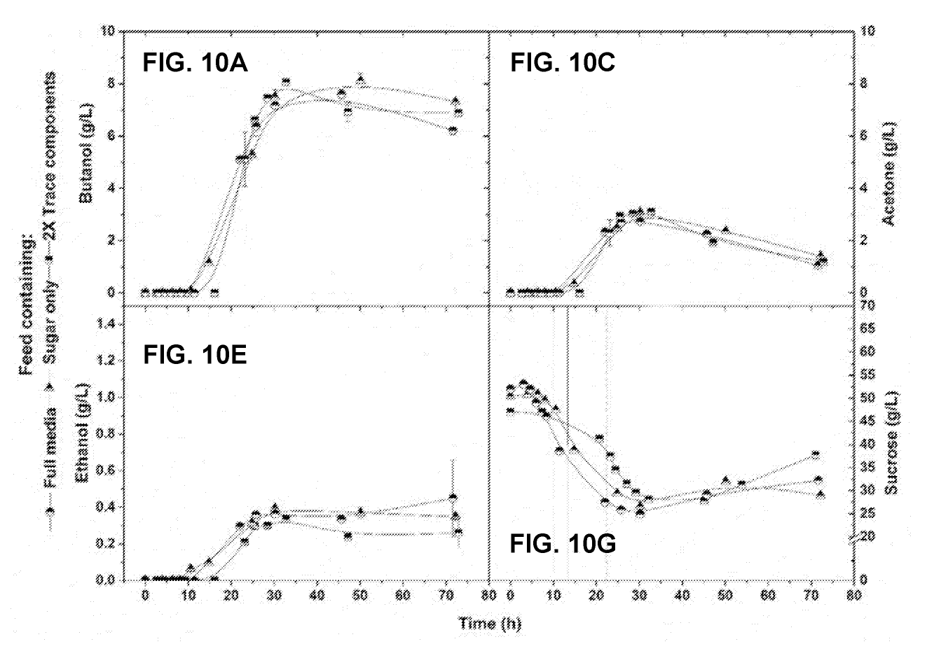

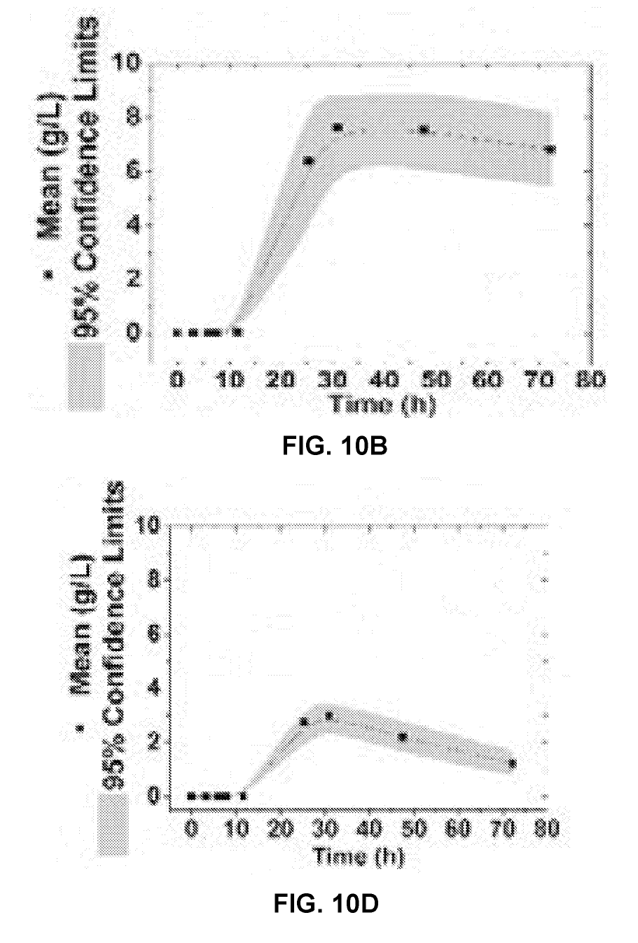

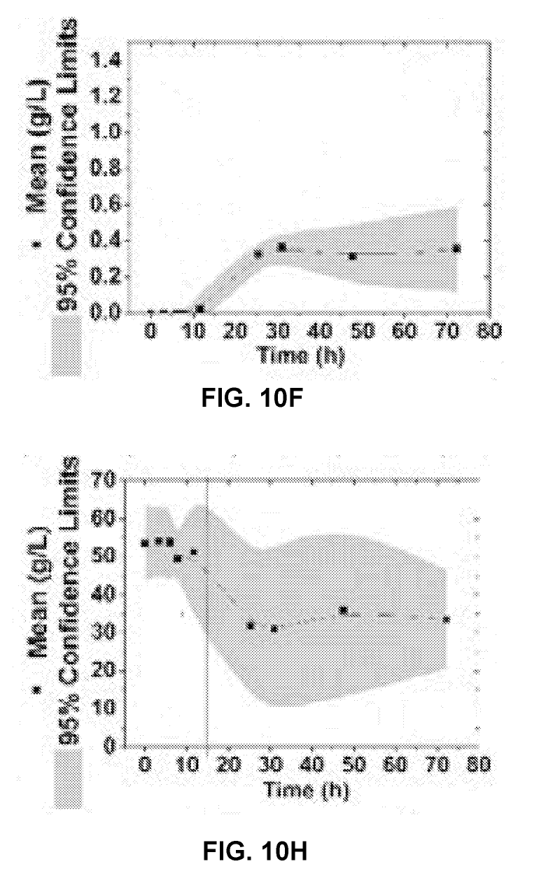

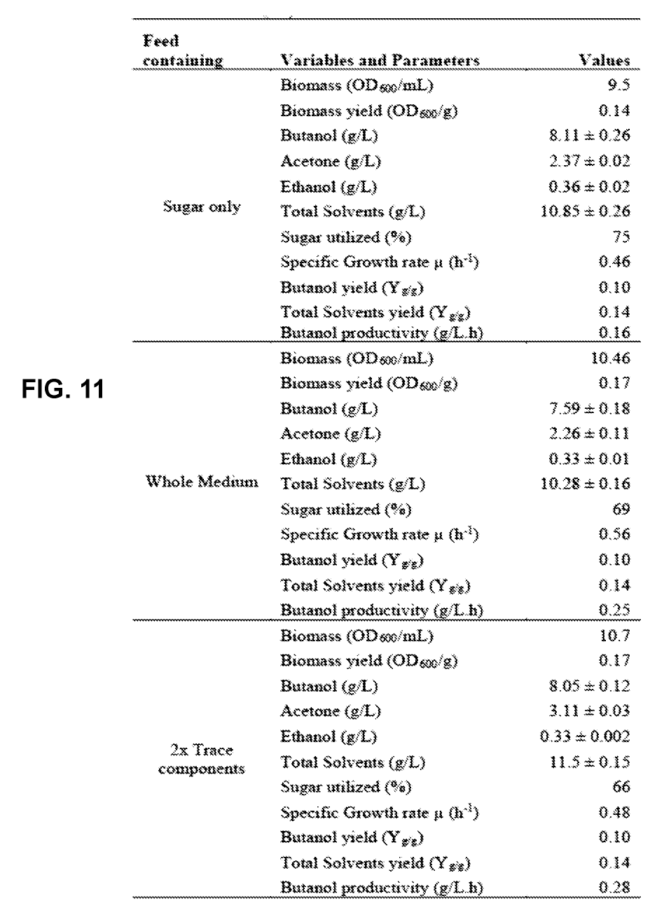

Real-time (in-line) fermentation gas monitoring reveals CO₂ and H₂ oscillations. A series of fed-batch fermentations of C. beijerinckii was performed while monitoring, in real-time (in-line), the evolving endogenous gasses. Interestingly, we observed in-phase, synchronous H₂ and CO₂ oscillations coinciding with late log-phase and the onset of solventogenesis (when H₂ and CO₂ reached ≈3% [v/v]) (FIGS. 1A-1D). These types of fluctuations are normally observed in feedback-loop controls as a response to metabolic pathway changes^{19,20}. Diauxic growth was evident from the decrease in the specific growth rate as H₂ and CO₂ resumed their accumulation (FIG. 9). Although we tested three feed compositions, (e.g. all while containing additional sucrose: (i) fresh complete medium; (ii) 2× trace components²¹; (iii) sucrose only) this had no effect on the gas oscillations. This suggested to us that neither organic carbon nor another medium component modulated this phenotype. Furthermore, the cells failed to utilize all the provided sugar, and its utilization varied significantly among experiments (FIGS. 10G-10H). In contrast, the kinetic and yield parameters for products and biomass did not vary significantly (FIGS. 10B, 10D, 10F, and FIG. 11). Nitrogen (F=12.48 L/h) was used as carrier/stripping for gas measurements. In batch (i.e. closed-system) without stripping CO₂ and H₂ accumulated in the head-space and reaches 48 and 23%, respectively, in batch (i.e. closed-system) (not shown).

It was previously observed that recirculating endogenous H₂ and CO₂ during butanol fermentation (for maintaining anoxic conditions) allows for more sugar consumption and acid generation¹⁵. Additionally, increased product biosynthesis has been shown when electrochemical bioreactors were cultured with C. acetobutylicum in complex medium along with CO₂²². Interestingly, the C-1 assimilation in bacteria is also a mechanism for redox balance, helping to sustain substrate uptake²³. Nevertheless, C-1 assimilation has not been previously described in Clostridium beijerinckii^{6,7}.

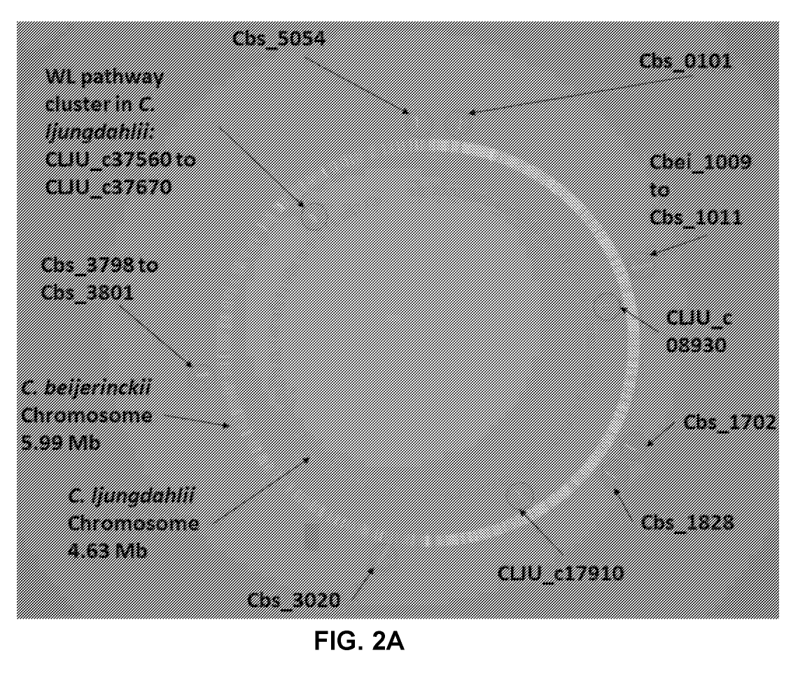

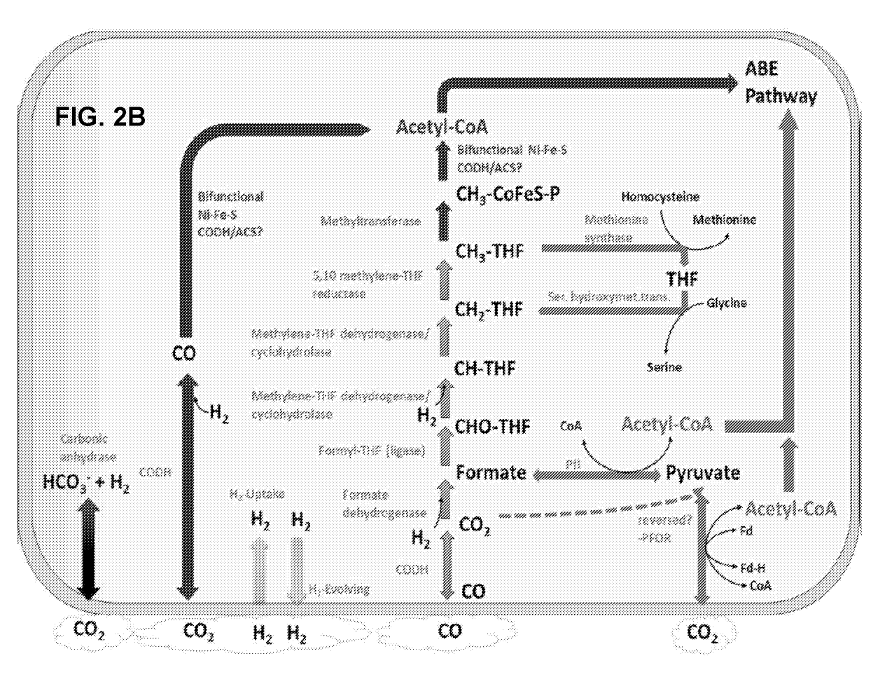

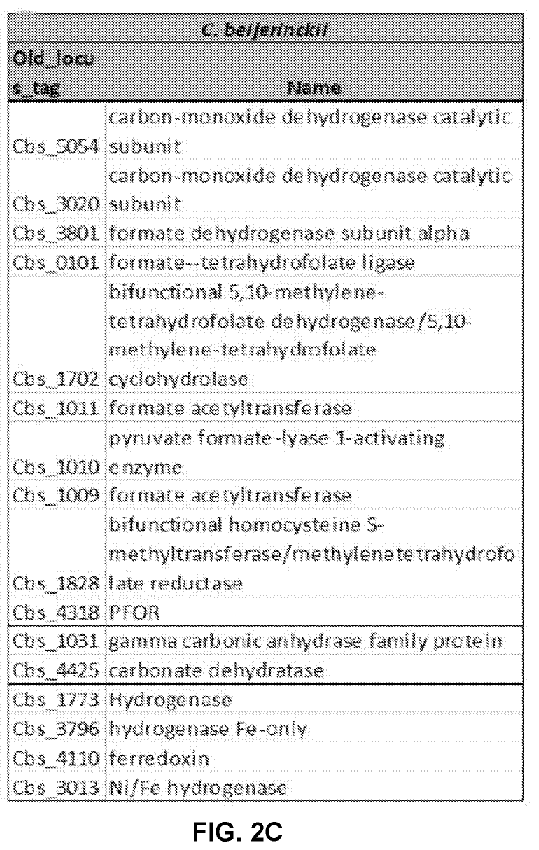

Genomic and indirect transcriptomic analysis indicates that C. beijerinckii has the genetic potential for C-1 assimilation. To explain the gas oscillations and their potential assimilation, the C. beijerinckii genome was examined by searching for genes related to C-1 assimilation, such as those associated to the WL or rPFOR/Pfl pathways^{2,5,7,24}. Open reading frames were found that putatively code for CO dehydrogenase (CODH) (Cbei_5054 and Cbei_3020), formate dehydrogenase and accessory genes (Cbei_3798 to Cbei_3801), formyl-THF ligase (Cbei_0101), methylene-THF dehydrogenase/cyclohydrolase (Cbei_1702) and methylene-THF reductase (Cbei_1828). The putative proteins encoded by these genes have high sequence identity to those of Clostridium ljungdahlii, the species most often utilized for ethanol generation from synthesis gas^{5,13} (FIGS. 2A-2B). However, as opposed to this species, C. beijerinckii does not contain annotated a gene coding specifically for an acetyl-CoA synthase. Furthermore, in C. ljungdahlii, most of the WL pathway genes are clustered, except a gene coding for a Ni—Fe—S containing CODH (CLJU_c17910), and a formate dehydrogenase (CLJU_c08930). The former is the main enzyme within the carbonyl branch of the WL pathway, and the final step of the methyl branch (or initial step, if CO is supplied). The latter initiates the methyl branch, allowing CO₂ capture into formate. Together, they lead to the generation of acetyl-CoA via an acetyl-CoA synthase. Conversely, C. beijerinckii contains the homologous genes scattered through its chromosome (FIG. 2A). Interestingly, the annotated CODH and the formate dehydrogenase from C. beijerinckii have 77.62 and 72.23% sequence identity, respectively, to the corresponding genes of C. ljungdahlii localized outside its WL cluster. In addition to these genes, C. beijerinckii contains Fe-only and NiFe-hydrogenases (Cbei_1773, Cbei_3796, Cbei_4110 and Cbei_3013) with similarities to those of C. ljungdahlii. In this species, along with H₂ generation, these enzymes have hydrogen uptake capabilities, providing extra reducing equivalents to its C-1-fixation pathway⁵.

C. beijerinckii also contains two putative Pfl-coding genes (Cbs_1009 and Cbs_1011), (both annotated as formate acetyltransferase, as is the case in Clostridium thermocellum [pflB, clo1313_1717)])², and a putative pyruvate formate-lyase activating enzyme gene (Cbs_1010). The proteins coded by Cbs_1009 and Cbss_1011, and Cbs_1010, have ˜63.5 and 44.4% sequence identity to those of C. thermocellum, respectively. This bacterium, while relying on a partial WL pathway (i.e. the methyl branch without the formate dehydrogenase), contains a reverse pyruvate ferredoxin oxidoreductase (clo1313_0673 and others) that combines acetyl-CoA and CO₂ to generate pyruvate, which is then transformed into formate and acetyl-CoA by Pfl². Interestingly, the C. beijerinckii pyruvate ferredoxin oxidoreductase (PFOR) (Cbs_4318) has 64.1% sequence identity to C. thermocellum rPFOR. The reverse reaction of PFOR has also been observed in other acetogenic and methanogenic bacteria, where this enzyme links the WL pathway and glycolysis²⁵. Additional genes related to the rPFOR/Pfl pathway are a serine hydroxymethyltransferase and a methionine synthase, both of which are also encoded in C. beijerinckii chromosome (Cbs_1868, and Cbs_3100, Cbs_2329 and Cbs_1401, respectively).

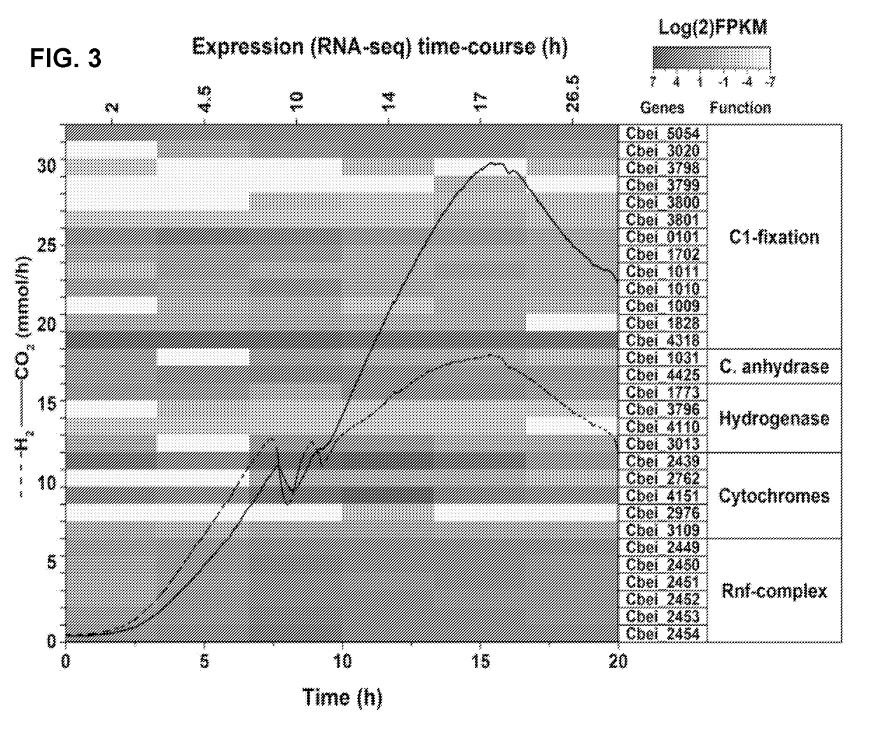

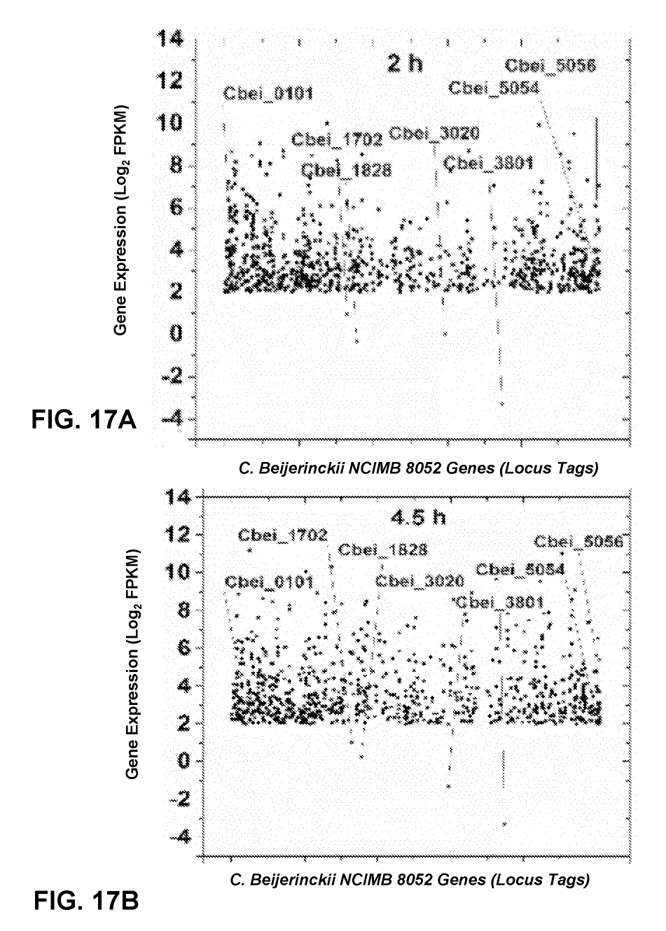

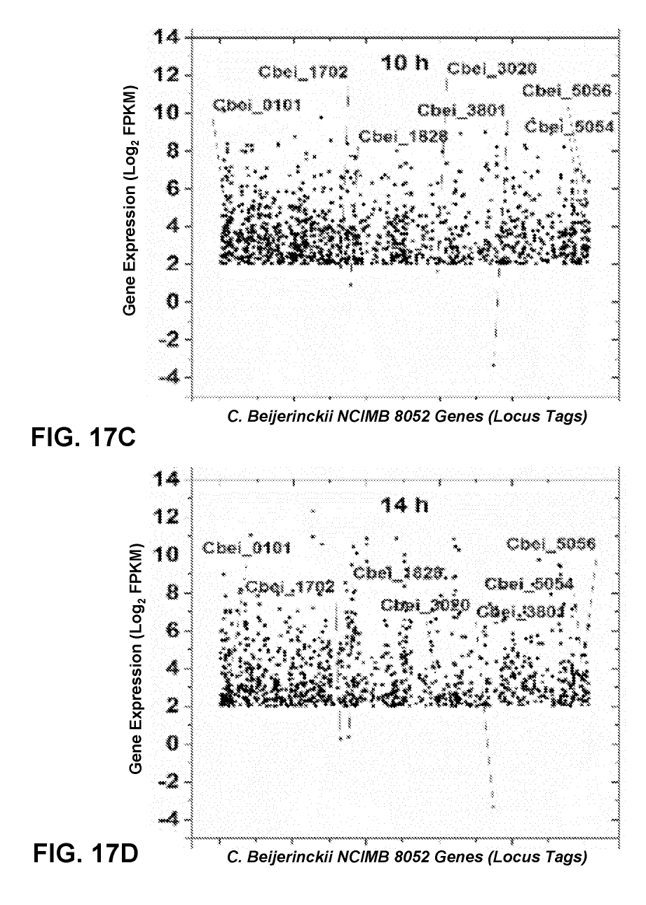

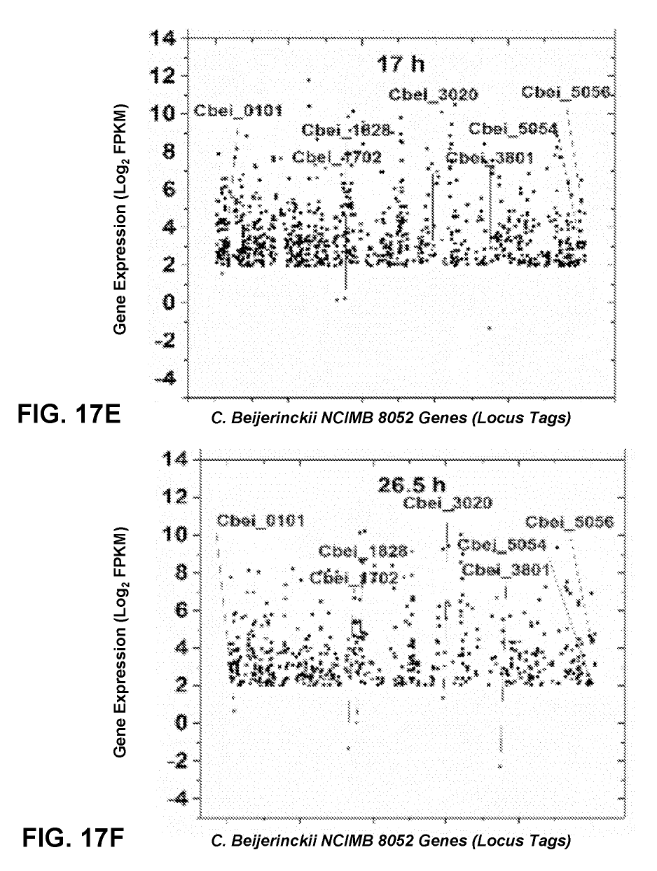

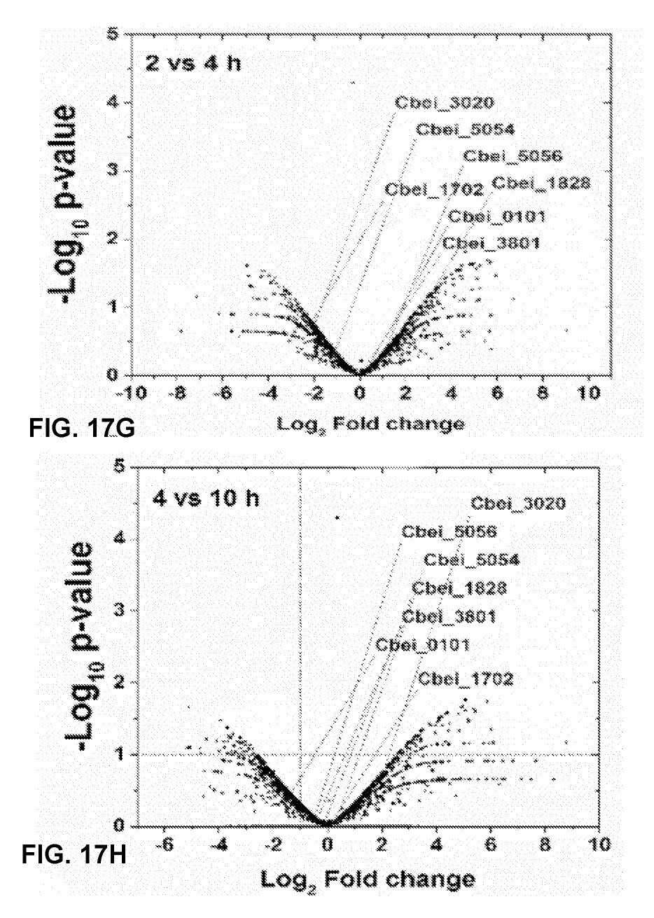

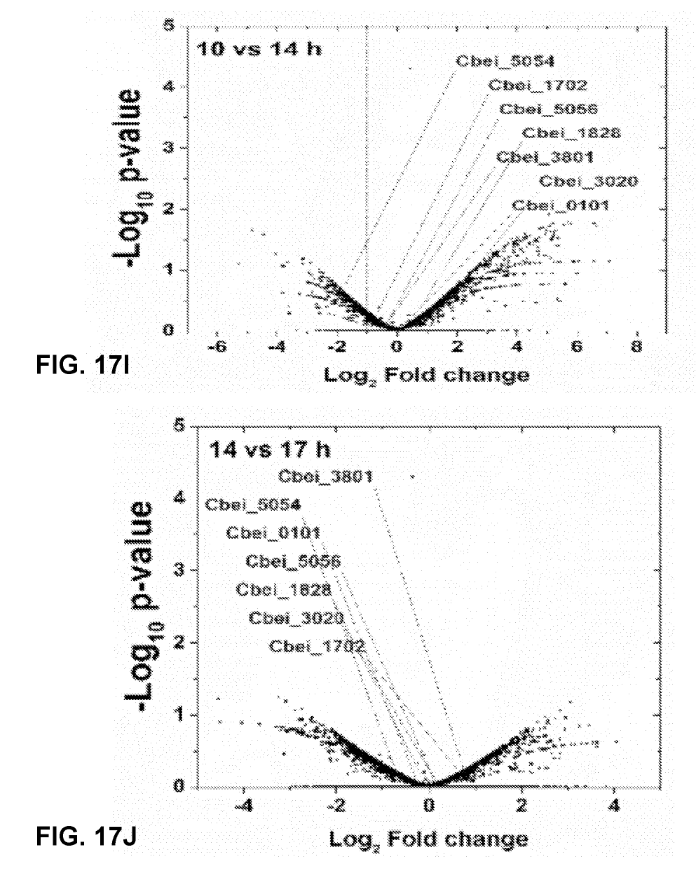

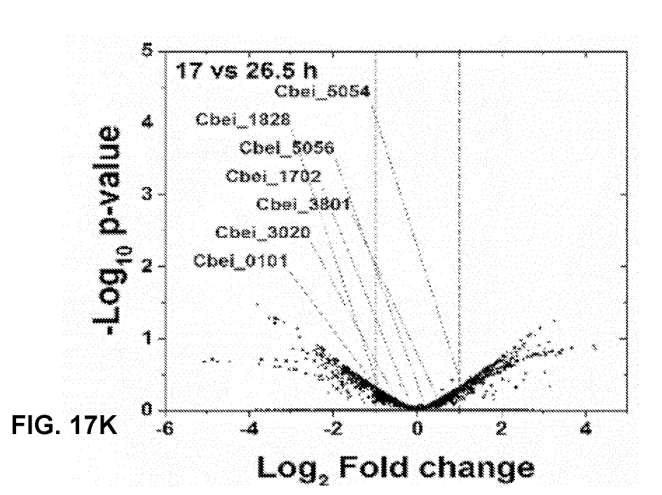

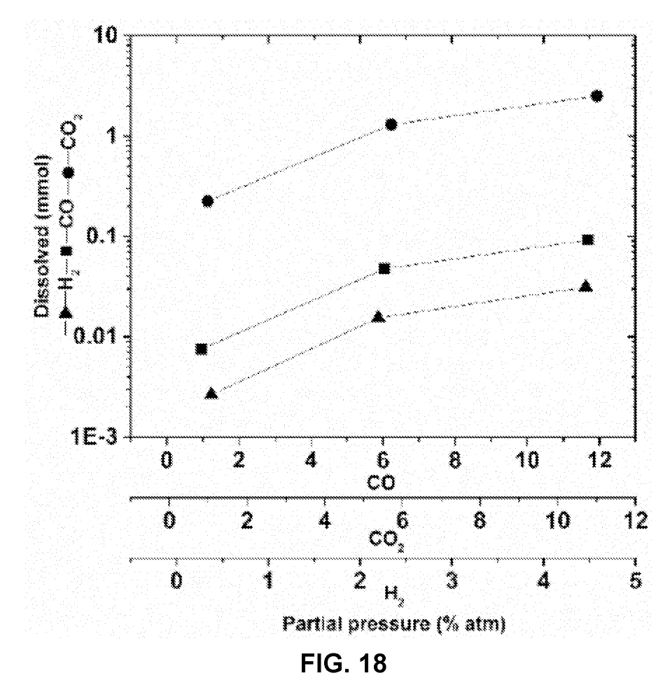

With these C-1 assimilation genes in focus, an analysis of publicly available transcriptomic data from batch cultures of C. beijerinckii²⁶ was conducted. An RNA-seq time-course experiment was previously reported by Wang et al²⁷ using cells growing in P2 medium sparged with pure nitrogen. After quality trimming and normalization for gene length and number of assembled reads, we found the putative genes required for C1-assimilation being expressed, either constitutively (Cbei_5054, Cbei_1828 and Cbei_4318) or differentially over time (Cbei_1702, Cbei_0101, Cbei_3801, Cbei_3794, Cbei_3798, Cbei_3799, Cbei_3800, Cbei_3020, Cbei_1010 and Cbei_1011) (FIGS. 3 and 17A-17K). After mapping above transcriptomic response to our gas oscillation data, expression changes were identified that coincided with this phenotype, indirectly pointing towards the re-assimilation of these gases (FIGS. 1A-1D and FIG. 3). Among these genes, formate dehydrogenase and its accessory genes showed the lowest expression in the evaluated experimental condition (i.e. low biomass and CO₂/H₂ concentration, and nitrogen atmosphere); however, increasing towards mid-log-phase, concurring with the time-point where CO₂ and H₂ accumulation is maximal (FIG. 3). These genes belong to the methyl-branch of the WL, and rPFOR/Pfl C1-assimilation pathways. Among the four annotated hydrogenase genes, two displayed the highest expression levels: the correlation between expression and hydrogen oscillation/evolution suggested that Cbei_3013 and Cbei_1773 are primarily used for H₂ uptake and H₂ evolution, respectively. Additionally, the genes encoding a carbonic anhydrase also showed expression (Cbei_4425/Cbei_1031). This enzyme allows for even more intracellular CO₂ availability²⁸. This in-silico analysis provided an indirect overview of the gas oscillations at the transcriptomic level, while suggesting that these gases may regulate the C1-assimilatory phenotype in C. beijerinckii. Similar gas-dependent behavior has been previously observed in cultures of acetogens Clostridium thermoautotrophicum and C. ljungdahlii, which contain a complete WL pathway^{28,30}. Clearly, C. beijerinckii has the genetic potential for inorganic carbon assimilation (FIG. 2B).

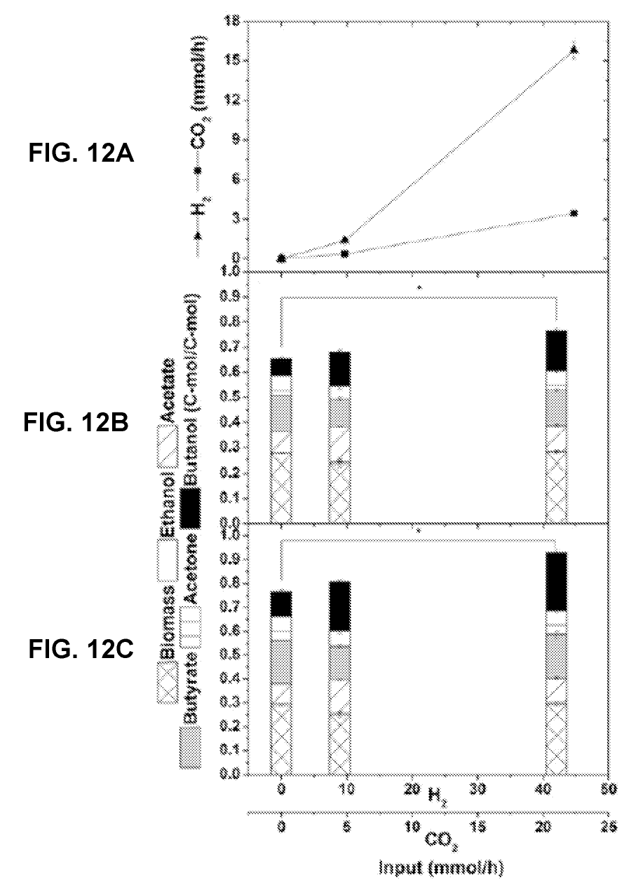

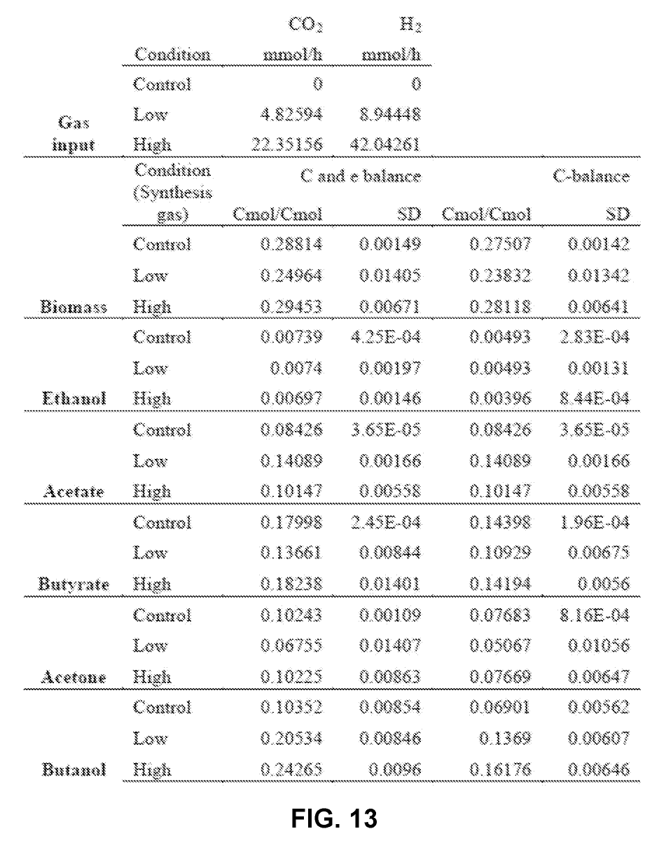

Functional evaluation shows inorganic carbon capture by C. beijerinckii. Interested in direct evidence of C-1 assimilation, we performed mixotrophic (sucrose 3% and fructose 1.5% [w/v]) chemostat fermentations (D=0.135 h⁻¹) while steadily sparging CO₂ and H₂ at high and low concentrations, balanced with nitrogen. We observed steady-state consumption of CO₂ and H₂ along with proportional increases of apparent product yields values above theoretical levels (FIGS. 12A-12C and FIG. 13). If sucrose and fructose were the only carbon and energy sources, apparent yields should have remained at or below the theoretical maximum (i.e. about 0.66% C-mol, as a result of one decarboxylation from C-3 pyruvate to the C-2 acetyl group of acetyl-CoA). The higher-than-maximum apparent yields indicated additional carbon assimilation that was only possible by inorganic carbon capture.

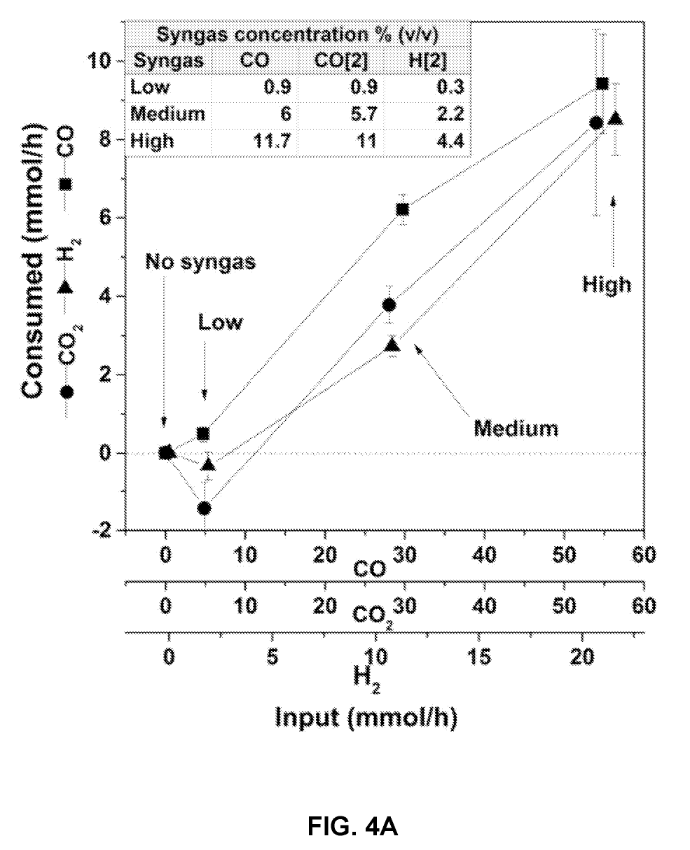

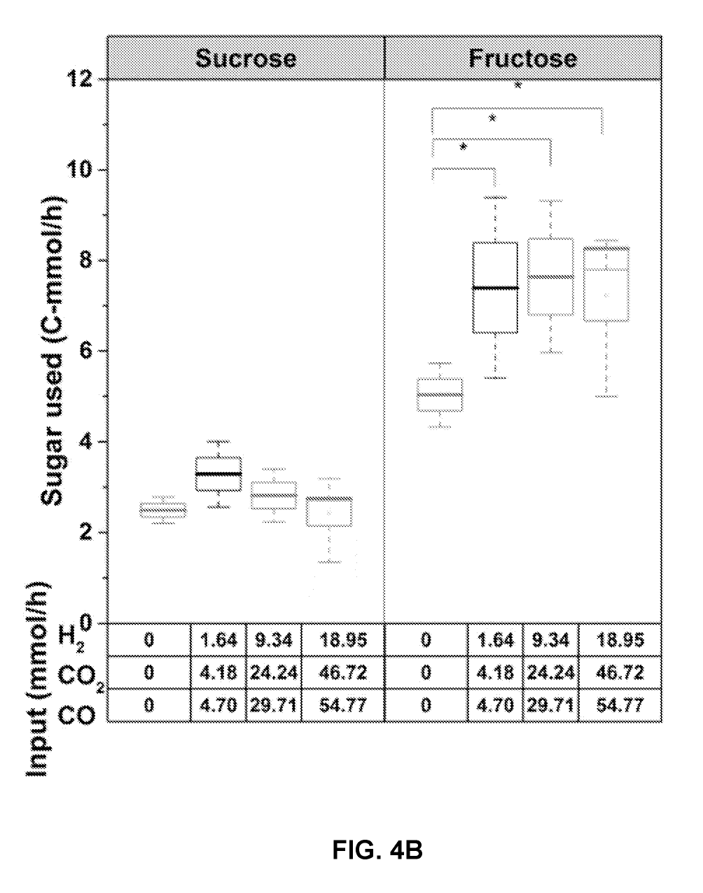

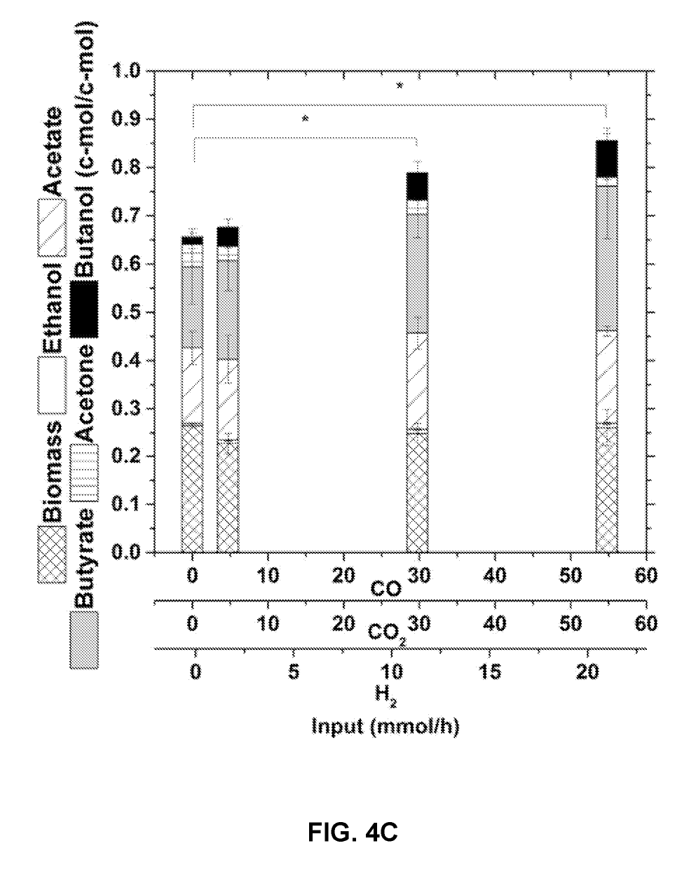

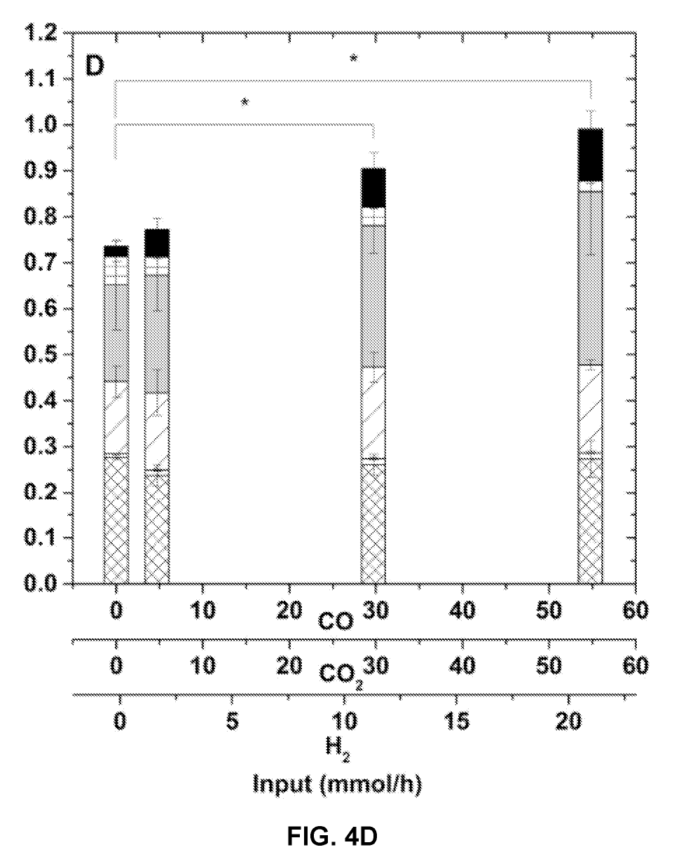

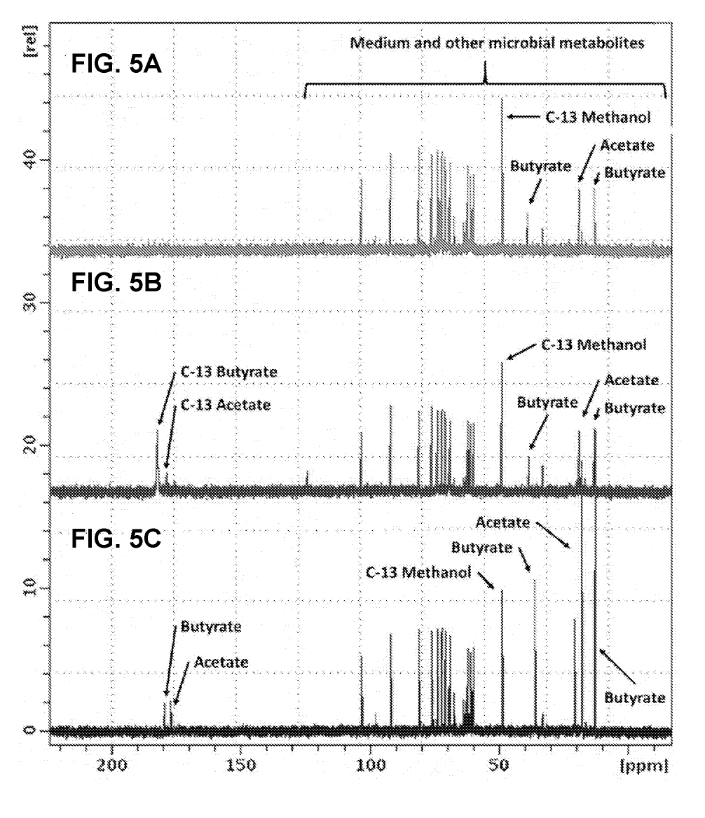

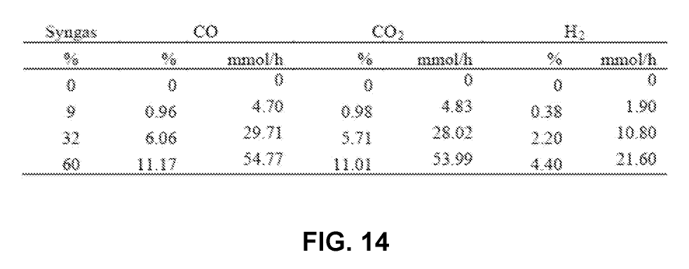

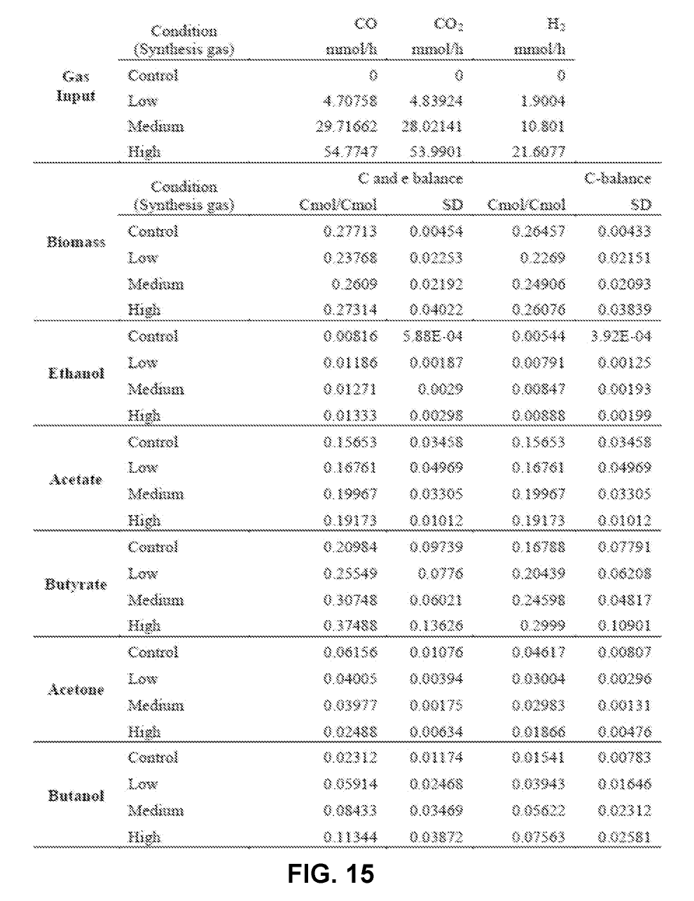

Considering current efforts to transform surplus synthesis gas into biofuels^{6,14}, we also sparged this gas at increasing step-wise concentrations (FIG. 14). Specifically, we sparged synthesis gas mixtures from low (9%), to medium (32%), to high (60%) concentrations, balanced with nitrogen (100% synthesis gas contained 20% CO, 20% CO₂, 10% H₂ and 50% N₂). At low concentration, C. beijerinckii oxidized CO, releasing H₂ and CO₂ as shown by the steady-state values (FIGS. 4A-4D and 15). Accordingly, there was a statistically nonsignificant difference in apparent C-mol yields and carbon-energy recovery balance compared to the control. Additionally, with increasing electron sink availability, the steady-state sugar utilization improved, resulting in higher product titers (FIG. 4B). Similar behavior has been observed by acetogens Clostridium thermoaceticum (Now Moorella thermoacetica)³¹, C. autoethanogenum, Rhodopseudomonas gelatinosa and also Carboxydothermus hydrogenoformans, according to the following reaction: CO+H₂O→CO₂+H₂, which is mainly used for redox balance when grown mixotrophicallyl^{2,23,32.} Therefore, in agreement with the transcriptomic analysis (e.g. overexpression of C-1 capture genes when CO₂/H₂ were maximal), this physiological behavior suggests these gases may be growth-limiting factors during C-1 assimilation by C. beijerinckii. Accordingly, when cultures were exposed to higher synthesis gas concentrations, higher-than-theoretical apparent C-mol yields and carbon/energy mass balances were detected with the concomitant increased consumption of H₂, CO, and CO₂, (FIGS. 4A, 4C, 4D). Specifically, at medium and high synthesis gas concentrations, 11 and 17% more carbon, and 19 and 27% more carbon and electrons, respectively, were recovered. Interestingly, butanol and butyric acid increased by 5.5- and 1.85-fold, respectively, while biomass did not change significantly, which is typical for C1-assimilation pathways such as WL, or rPFOR/Pfl. To confirm gas assimilation into products, steady-state cells (D=0.135 h⁻¹) were collected growing under high synthesis gas concentration and cultured them in batch conditions in presence of pure ¹³CO₂ in the headspace as tracer. After 48 h of incubation and NMR analysis, peaks at about 178 and 180 ppm revealed the presence of ¹³C-labeled acetate and butyrate, respectively (FIGS. 5A-5C). These peaks were likely from the enriched ¹³C quaternary carbon of these compounds. This was confirmed by adding acetate and butyrate standards (12.8 and 15 g/L, respectively), and observed an increase of peaks at 178 and 180 ppm (quaternary Cs of acetate and butyrate), at 22 (primary C of acetate), and 14 and 40 ppm (primary, secondary (also at 14 ppm), and tertiary Cs of butyrate, respectively). This is a typical spectrum of unlabeled compounds, where quaternary Cs are difficult to detect, as seen in the control in panel A (FIG. 5A). Overall, this provides direct evidence of C-1 assimilation by C. beijerinckii.

Although butanol is the main target in ABE fermentation and the proportion of total carbon in the form of n-butanol increased by 92%, butyric acid is also a value-added product and can be re-assimilated into n-butanol through multi-stage fermentations^{21,33}. The generation of C-4 compounds, such as butyric acid and butanol, require more NADH than C-2 compounds (such as ethanol)¹⁷, underscoring the cells emphasis in recycling electrons.

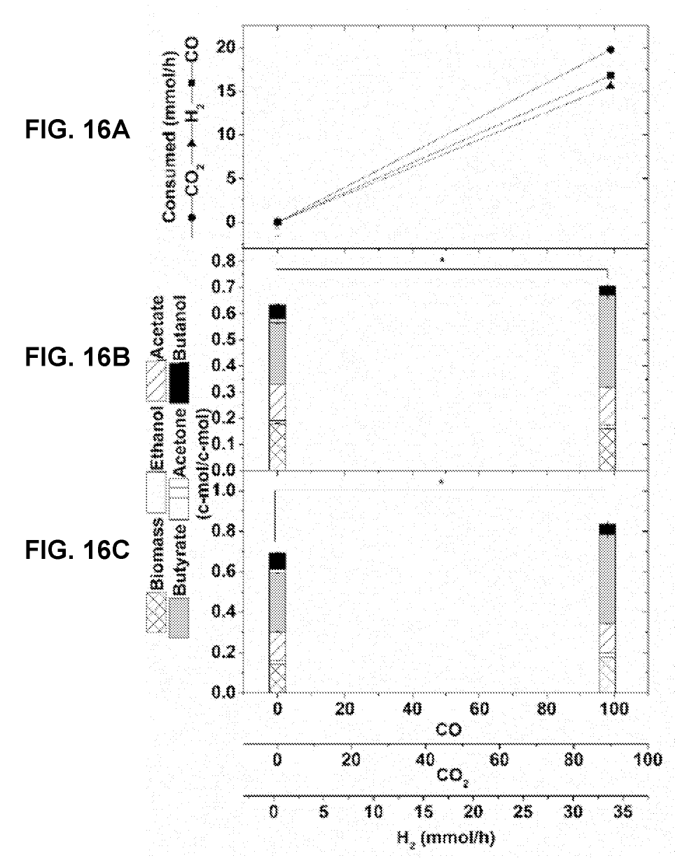

The rate of gas assimilation, larger than the saturation values in each condition, also indicated biological activity (FIG. 4A and FIG. 16A-16C). Higher synthesis gas concentrations were also tested (up to 100%, containing 20% CO, 20% CO₂, 10% H₂ and 50% N₂), showing carbon capture but generating lower yields, possibly due to carbon monoxide poisoning³⁴ (FIG. 18). This indicates that the fermentation working-window for mixotrophic capture of synthesis gas by C. beijerinckii is between 30 and 60%. These values are lower than the working conditions for cultures of C. ljungdahlii and C. carboxidivorans, also suggesting that the enzymatic affinity for carbon-capture of C. beijerinckii may be higher^{13,14}.

Batch fermentations of C. beijerinckii were also performed under a continuous flow of high synthesis gas concentration as sole carbon and energy source. Transient cell proliferation and CO assimilation was observed (not shown). However, cell growth and gas assimilation stopped in early exponential growth phase, as the cells initiated sporulation. As a result, no products were detected.

Transcriptomic Analysis of the Partial WL and rPFOR/Pfl Pathways in C. beijerinckii.

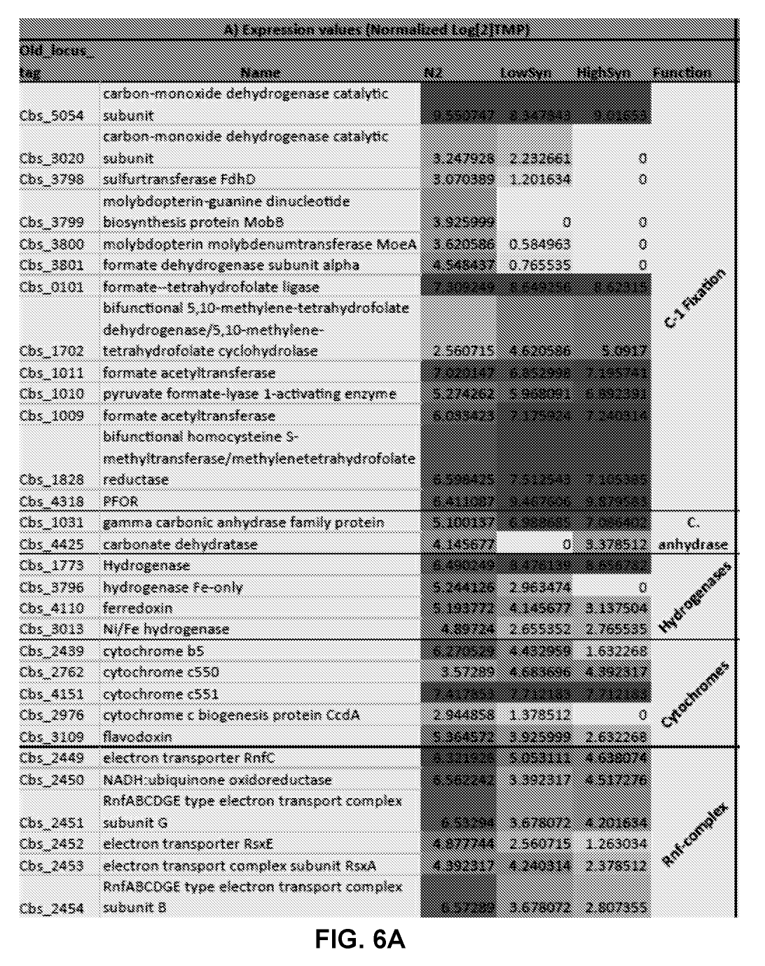

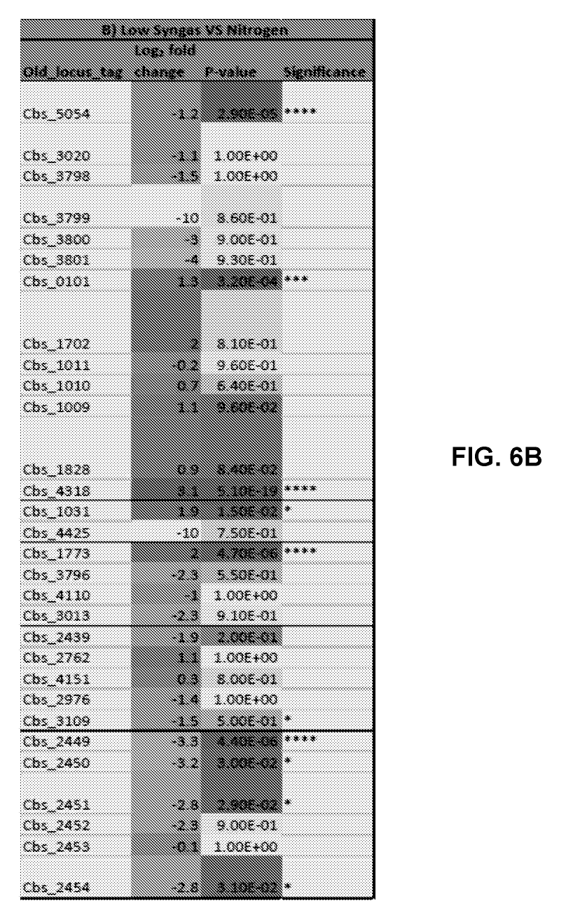

To complement the time-course transcriptomic data previously described, a RNA-seq experiment was performed using chemostat cultures (D=0.135 h⁻¹) of C. beijerinckii SA-1, continuously sparged either with nitrogen (control), low or high synthesis gas. FIGS. 6A-6C show constitutive expression of each gene under N₂ conditions (normalized to transcripts per million [TPM]), but differentially expressed under both synthesis gas conditions. Under low concentration of synthesis gas, there was a significant overexpression of a putative formate-THF ligase (Cbs_0101), which belongs to the WL pathway, a PFOR (Cbs_4318), a carbonic anhydrase (Cbs_4425), and a hydrogenase (Cbs_1773). Conversely, there was a repression of a putative CODH (Cbs_5054), a gene that belongs to the formate dehydrogenase complex (Cbs_3799), a flavodoxin (Cbs_3109), and several genes that putatively code for the Rnf-complex (Cbs_2449/54). Under high concentration of synthesis gas, there was a significant overexpression of the same genes under low synthesis gas, and a Pfl-activating enzyme gene (Cbs_1010), which belongs to the rPFOR/Pf pathway. However, under that condition, the formate dehydrogenase complex, a CODH (Cbs_3020), a hydrogenase (Cbs_3796), and a cytochrome c biogenesis coding protein (Cbs_2976) were completely shut down. Overall, this transcriptomic data suggests that C. beijerinckii constitutively expresses its putative genes associated to C-1 capture, by preferentially activating those belonging to the rPFOR/Pfl pathway. Specifically, when cultured under synthesis gas, the repression of the formate dehydrogenase, and the overexpression of PFOR and Pfl suggest that CO₂ is assimilated via rPFOR/Pfl pathway (and the carbonic anhydrase), as observed with C. thermocellum².

Nitrite as an Electron Sink for Energy Conservation.

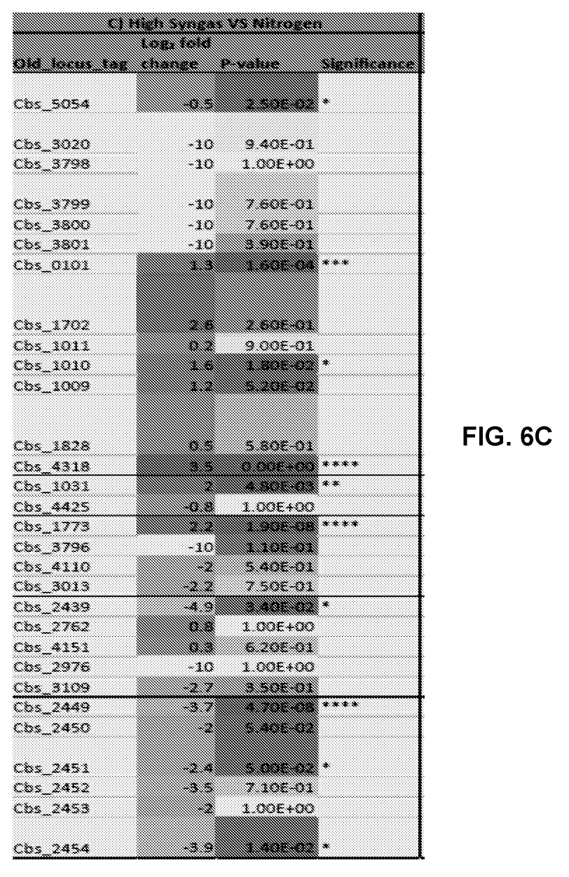

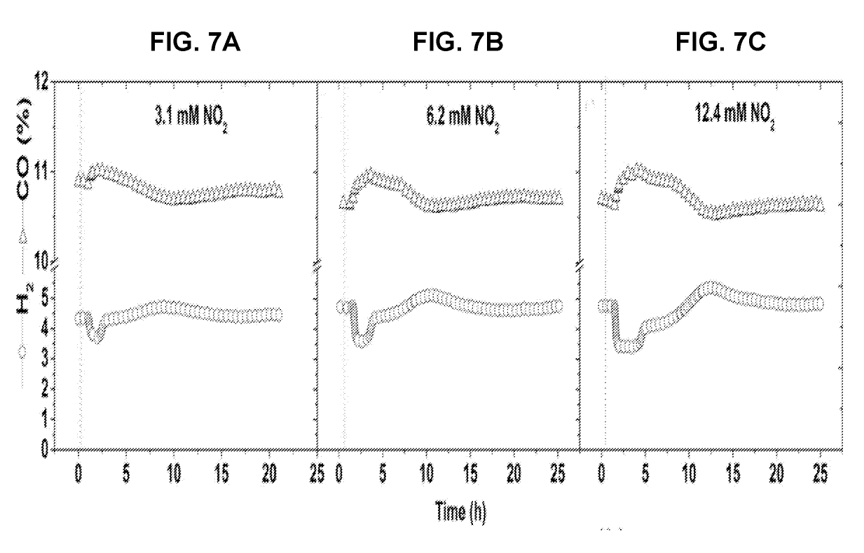

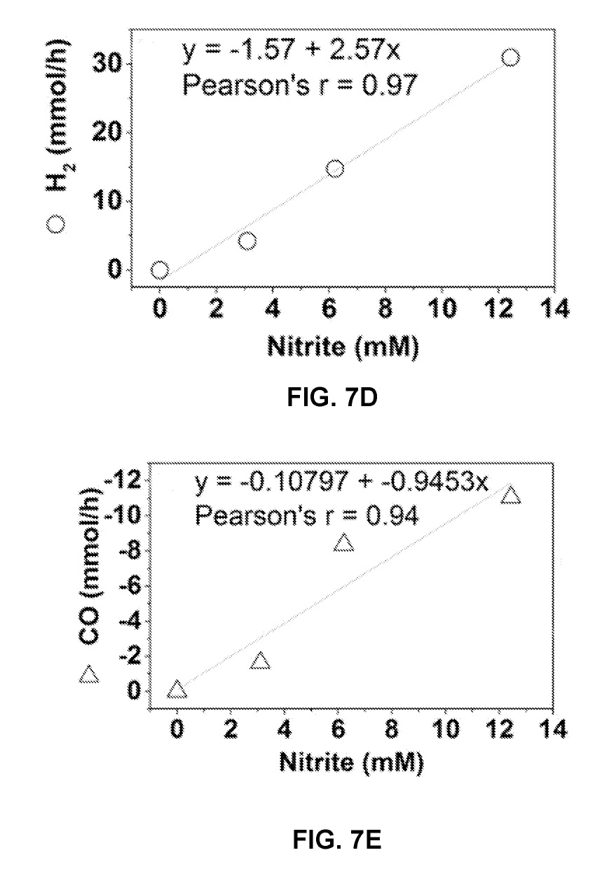

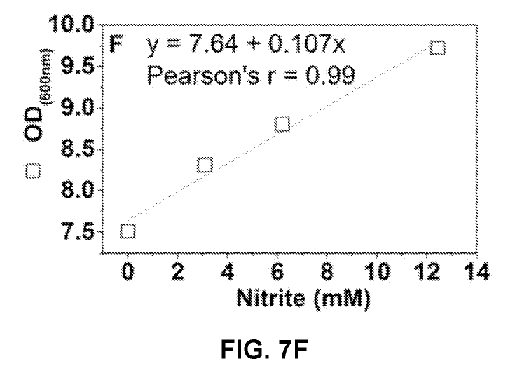

Under mixotrophic growth, the C-1 assimilation pathways operate mainly for electron recycling^{2,23,35}. Consequently, an alternative way to demonstrate an active pathway is to inhibit CO assimilation by providing an alternative and preferred electron acceptor. Both nitrate and nitrite are known to have this effect on CO assimilation by acetogenic bacteria^{36,37}. To test this hypothesis in C. beijerinckii, chemostat pulse experiments were performed under high synthesis gas concentration (e.g. about 60% [v/v]). Interestingly, nitrate showed no effect on C. beijerinckii. However, less-reduced nitrite partially inhibited CO assimilation (1 mol per mol of NO₂), while increasing hydrogen consumption (2.5 mol of H₂ per mol of NO₂). Additionally, biomass increased proportionately, shifting the pathway from catabolism to anabolism (FIGS. 7A-7F). Both electron acceptors have also been shown to increase biomass in acetogens C. thermoautotrophicum and Moorella thermoacetica^{36,37}. The H₂-dependent CO₂, or CO assimilations are thermodynamically unfavorable as they do not generate a gain in ATP³⁵. Thus, nitrite reduction is preferred as a less expensive way to recycle electrons. As such, the nitrite reductase reaction requires only electrons, in the form of hydrogen and reduced ferredoxin (i.e. not ATP). C. beijerinckii contains a putative ferredoxin-nitrite reductase (Cbei_0832), likely responsible for the observed phenotype, which also unveils this species as a facultative nitrite dissimilator.

Transcription of Alternative Energy-Conservation Genes.

Considering the poor energetics of the C-1 assimilation pathways, autotrophic bacteria rely either on substrate-level phosphorylation or on chemiosmosis for ATP synthesis^{7,23,35}. Examples of the latter include cytochromes, Na⁺ pumps, or the Rnf-complex, whereby acetogens generate an ion gradient for energy generation through ATP-synthases. B-type cytochromes are responsible for H⁺-dependent ATP generation, and can be coupled to a membrane-bound methylene-THF reductase³⁸. C. ljungdahlii contains a Rnf-complex but not cytochromes^{5,39,40}. Interestingly, the C. beijerinckii genome encodes cytochromes (also involved in nitrite reduction⁴¹) b-type (Cbei_2439), c550 (Cbei_2762), c551 (Cbei_4151), c biogenesis protein (Cbei_2976), cytochrome-bound flavoproteins (Cbei_3109), and also genes coding for the Rnf-complex (Cbei_2449-2454). Additionally, the methylene-THF reductase of C. beijerinckii is predicted⁴² to contain transmembrane domains. The transcriptomic analysis of the publicly available RNA-seq data²⁷ showed high expression of all these energy-conserving genes, especially the Rnf-complex (FIG. 3). In line with this observation, the transcriptomic analysis of chemostat cultures of C. beijerinckii shows constitutive expression of these genes under nitrogen exposure, and a modest repression under low and high concentrations of synthesis gas (FIGS. 6A-6C). Since sporulation prevents C. beijerinckii to growth autotrophically, these chemiosmotic mechanisms are potentially useful only during mixotrophic growth. Similarly, C. ljungdahlii requires the Rnf-complex when cultured mixotrophically⁴⁰.

Discussion

The variability on apparent product yields reported in the literature and the empirical records of microbial solvent production, demonstrated the need for a deeper study of the evolving gas-phase as signals for overlooked pathways. It has been shown that C. beijerinckii captures inorganic carbon and hydrogen under mixotrophic conditions; increasing apparent product yields above theoretical heterotrophic values. Among the putative WL pathway genes, C. beijerinckii does not contain annotated an acetyl-CoA synthase, but its CODHs have Fe-S and Ni—Fe-S metal centers, which are typical of bifunctional CODH/acetyl-CoA synthasee⁴³⁻⁴⁵. However, it is likely that this enzyme in C. beijerinckii does not lead to acetyl-CoA synthesis, and thus autotrophic growth. As has been recently shown, a mutant strain of C. ljungdahlii with a SNP (single nucleotide polymorphism) in its CODH gene located in its WL cluster (i.e. the one with lower sequence identity to that of C. beijerinckii, and associated to a acetyl-CoA synthase) loses its autotrophic phenotype, even when its CODH with similarity to C. beijerinckii was intact. Nevertheless, C. beijerinckii contains the genetic potential for an active rPFOR/Pfl-based C-1 capture, including an additional formate dehydrogenase, not present in C. thermocellum².

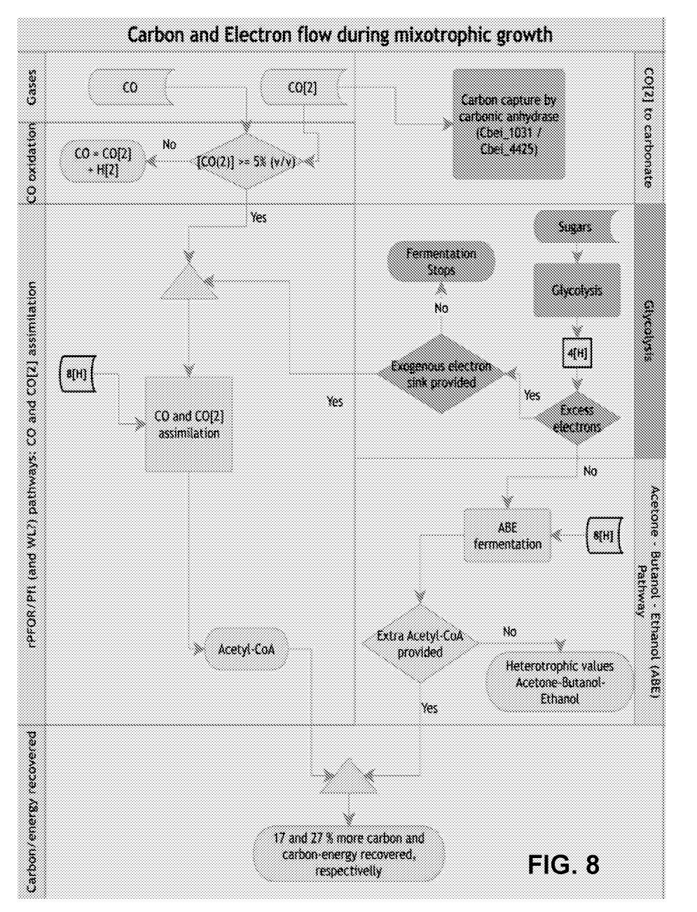

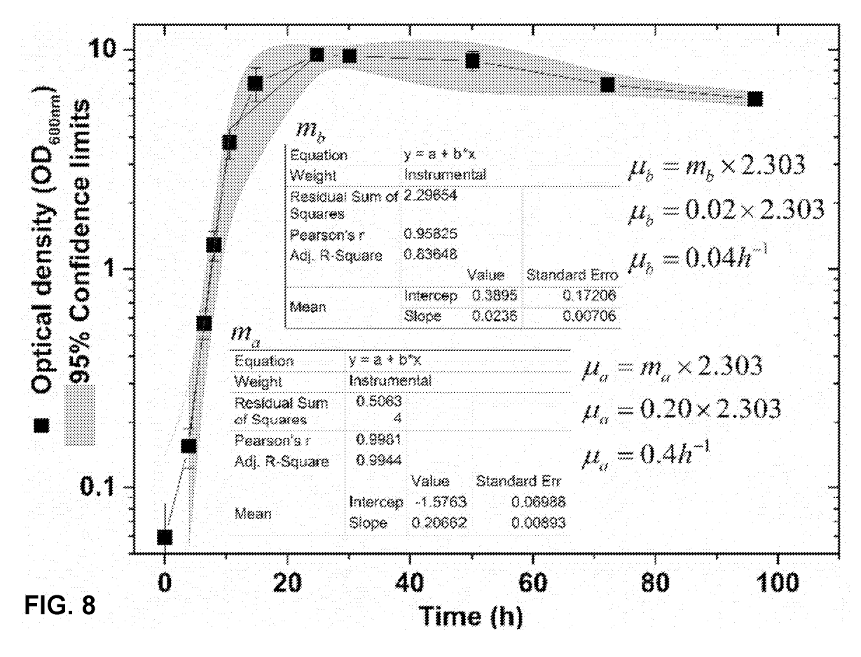

Based on our physiologic data, a logic model was constructed to explain the carbon-electron flow during mixotrophic growth of C. beijerinckii cultures (FIG. 8). In the presence of CO and CO₂, there are three possible paths for carbon capture: 1) CO₂ to carbonate through carbonic anhydrase, or 2) CO oxidation to generate CO₂+H₂, if the CO₂ in the gas-phase is <5% (v/v); or finally, 3) C-1 assimilation into acetyl-CoA, if CO₂>5%. Simultaneously, supplied sugars proceed to glycolysis. In the absence of an electron bottleneck, ABE-fermentation utilizes all the sugar-derived carbon and electrons. Otherwise, and if no external electron sink is provided, fermentation stops, limiting sugar utilization. In the presence of an external electron sink (such as CO/CO₂), #2 or #3 takes place. If #3 takes place, the extra acetyl-CoA generated, along with the still-running ABE-pathway, leads to 17 and 27% more carbon and carbon-energy recovered, respectively. Moreover, this physiological capability improves product titers by increasing sugar utilization. However, under these conditions herein about 86 and 100% total carbon and carbon/electron were observed to be recovered, respectively.

Mixotrophic C-1 assimilation was previously shown by cultures of acetogen C. ljungdahlii, whereby exogenous CO₂ gas increases carbon recovery⁴⁷. The discovery of the same phenotype by cultures of C. beijerinckii has important implications in our understanding of the biology of this industrial butanol-producer, and adds a new alternative for greenhouse gas-capture. Indeed, C. beijerinckii stands out among traditional acetogens and solventogenic species because: (i) it contains genetic elements for cytochromes and the Rnf-system; (ii) it contains genes that code for catalytic enzymes that belong to the WL (except acetyl-CoA synthase) and rPFOR/Pfl pathways; and (iii) the synchronous H₂/CO₂ oscillation is an example of a natural integrated oscillator, that can potentially be used for feedback controls in biosensors^{48,49}. The approach for in-line endogenous gas monitoring shows that it can readably be utilized to uncover new pathways, or potentially even survey a culture (or consortia) for volatile metabolic signatures, in real-time.

Materials and Methods.

### Organisms:

Clostridium beijerinckii SA-1 (ATCC 35702)²⁶ was obtained from the American Type Culture Collection (ATCC). Its identity was verified by PCR amplification and sequencing of the 16S rRNA gene using the prokaryotic 16S rDNA universal primers 515F (5-GCGGATCCTCTAGACTGCAGTGCCA-3 (SEQ ID NO: 1) and 1492R (5-GGTTACCTTGTTACGACTT-3 (SEQ ID NO: 2)).

Bacterial Medium and Inoculum Preparation:

C. beijerinckii stocks were activated as previously described¹⁸ and were grown in a previously designed medium²¹. The base components were autoclaved and the sugar (6% w/v sucrose) and trace components were added aseptically to the medium reservoir by filtration (0.22 μm). The inocula were prepared as consistently performed by our lab⁵⁰. Exact fermentation conditions are detailed in the Main Text section.

Bacterial Culture Conditions:

Growth experiments were performed in fed-batch or chemostat modes of operation in a 2-Liter Biostat® B plus fermenter equipped with controllers for pH, temperature, agitation, and gas mass-flow (Sartorius BBI Systems, Germany). The temperature was set at about 37° C., agitation speed at about 250 rpm, and pH about 6.5 by the automated addition of about 0.5 N KOH or about 25% (v/v) H₃PO₄, into a working final volume of about 1,400 mL of culture for fed-batch or about 700 mL for chemostat. The fed-batch fermentations were started with about 6% (w/v) sucrose, and about 400 mL containing about 80 g of the same sugar were added at constant feed rate (about 0.08 mL/h) to reach a final concentration of about 100 g/L (w/v). The initial volume was about 1 L and final about 1.4 L. Exact feed components and times of feed start are detailed elsewhere herein. For the chemostat experiments the conditions were identical as described for fed-batch except the carbon and energy source were about 3% (w/v) sucrose and about 1.5% fructose. Once the cells reached exponential phase under sparged pure nitrogen (OD600 nm about 1), the feed and harvest flow were initiated and adjusted to a dilution rate of about D=0.135 h⁻¹. Exact sparged gas compositions are detailed elsewhere herein, steady-state conditions were verified for each condition and at least three retention times were allowed before sampling was initiated. Three samples at each steady-state condition were obtained from at least one retention time intervals. The discrete ratios of continuous gas streams were always sparged at about 12.48 L/h. Different gas-phase conditions, from pure nitrogen gas to increased synthesis gas concentrations, were achieved by modifying the mix ratios between synthesis gas and nitrogen using two mass flow controllers; the exact concentrations tested are detailed in the Results section (and FIG. 13). Inlet and exhaust gases in the gas-phase (O₂, N₂, CO, CO₂, H₂, and Ar) were monitored and recorded in real-time using in-line O₂/CO₂ and H₂/CO EasyLine continuous gas analyzers, model EL3020 (ABB, Germany), and a Pfeiffer OmniStar quadrupole mass spectrometer. Biomass proliferation in the fermentation tank was monitored and recorded using an in-line biomass sensor (Fundalux, Sartorius, BBI Systems, and Germany) and also by discrete measurements of the optical density (OD_{600 nm}) on a digital spectrophotometer (SmartSpec Plus, BioRad, USA). Dry weight concentration was obtained by filtering a portion of sample using vacuum suction through a 0.2-μm-pore-size filter of known mass (mixed cellulose esters; EMD Millipore, Germany); the filter was then dried at about 60 to about 70° C. for about 7 days and reweighed until constant weight.

Sample Analysis.

Sucrose, fructose, acetic and butyric acid were quantified with a high-performance liquid chromatograph (HPLC) under isocratic conditions at about 65° C., and a mobile phase of water at about 0.5 mL/min flow rate using a Supelcogel™ Ca column (about 300 mm×about 7.8 mm, Supelco™ Analytical, Bellefonte, Pa., USA) coupled to a refractive-index detector. Solvents (acetone, butanol and ethanol) were separated in a gas-chromatograph (GC) SS Porapak Q 80/100 column (OV, Marrietta, Ohio, USA) in a GC (GC-8A) fitted with a flame ionization detector (FID) (Shimadzu Corporation, Kyoto, Japan), using about 200 kPa of nitrogen as the mobile phase with an injection temperature of about 220° C. and a column temperature of about 140° C.

C-13 labeled-CO₂ experiments were performed using 50 mL of culture was collected from the steady state (D=0.135 h⁻¹) under high synthesis gas concentration (about 11.17 CO, about 11.01% CO₂ and about 4.40% H₂ [v/v]), depicted in FIGS. 4A-4D. Cultures were incubated for about 48 h in sealed about 250 mL serum bottles containing about 100% C-13-labeled CO₂ in the head space, at about 37° C., 200 rpm. For analysis, samples were sent to the NMR-Center of the Chemistry Department at NC State University. 13-C NMR spectra were carried out using a Bruker DRX-500 spectrometer equipped with a 5 mm ITD probe, maintaining the temperature constant at about 298K and the acquired process data were measured with the same parameter. The “zgig” pulse sequence was used. The data were acquired with 2600 FIDs. The internal standards used were C-13 methanol, butyric and acetic acid (0.081, 9.051 and 7.68 μg, respectively), into about 600 μL of sample, diluted into about 10% D₂O).

Proteins Sequence Identity Analysis:

Protein sequence identity were performed as previously described⁵¹.

RNA-Seq Analysis from Wang et al:

The sequence reads from the transcriptional profiling experiment of Wang et al²⁷ were downloaded from the NCBI Sequence Read Archive (SRA045799) and imported into the Cyverse Collaborative Discovery Environment⁵². The sequence reads were quality filtered with the trimmomatic program⁵³ using the trimmers, “LEADING:5 TRAILING:5 SLIDINGWINDOW:4:15 MINLEN:36”. Two independent platforms were subsequently used to analyze these normalized data, Cyverse Discovery Environment and Geneious v9 (Biomatters Ltd., New Zealand), while aligning the sequences to the C. beijerinckii NCIMB8052 genome (GenBank accession CP000721.1). In Cyverse, the sequences were aligned with tophat2⁵⁴ using the default parameters, while differences in transcript abundance were determined using the Cuffdiff program, which is part of the Cufflinks software package⁵⁵. The analyses in Geneious were performed using default parameters.

Transcription Expression Analysis:

To complement the time-course transcript expression analysis of RNA-seq experiments previously described, we performed chemostat cultures (D=0.135 h⁻¹) of C. beijerinckii SA-1 growing in presence of sparged N₂, low, or high synthesis gas (FIGS. 12A-12C). For each condition cultures growing in steady-state were harvested (about 10 mL) and immediately flash-freeze and sent for RNA extraction and sequencing at the Microbiome Core Facility at the University of North Carolina Chapel Hill, N.C.

RNA Isolation:

RNA from bioreactor-derived bacteria was isolated using PowerMicroBiome RNA isolation kit from MO Bio Laboratories (San Diego, Calif.). Briefly, the bacteria pellets were combined with lysis buffer and glass beads. Subsequently they were lysed for about 5 minutes in Qiagen TissueLyser II (Valencia, Calif.) at about 30 Hz. Further, the process included inhibitor removal step and standard on-column purification was carried out according to manufacturer's instructions. RNA purification included on-column DNAse treatment for about 15 minutes at room temperature. Subsequently RNA concentration and quality were determined by RNA electrophoresis on Agilent bioanalyzer (Santa Clara, Calif.).

rRNA Removal and Library Preparation:

rRNA was removed using Illumina Ribo-Zero Gold Bacteria Kit (San Diego, Calif.), according to manufacturer's instructions. Briefly, the rRNA-specific magnetic beads were washed off the storage buffer and were mixed with about 500 ng of total sample RNA. Subsequently, rRNA removal solution was added and samples were incubated for about 10 minutes at about 65° C. Finally, samples were placed on magnetic stand for about 15 minutes in room temperature and coding RNA was aspirated after which it was immediately preceded to mRNA library preparation protocol. Illumina TruSeq Stranded mRNA Library Prep Kit (San Diego, Calif.), was used according to manufacturer's instructions. Briefly, RNA was mixed with Fragment-Prime mix and incubated at about 94° C. for about 8 minutes and then it was immediately subject to first strand and second strand cDNA synthesis reactions, respectively, followed by 3′ end repair, adenylation and adapter ligation. After adapter ligation, the libraries were enriched by polymerase chain reaction using the following thermal cycling conditions: about 98° C. for about 30 s, followed by about 15 cycles of about 98° C. for about 10 s, about 60° C. for about 30 s and about 72° C. for about 30 s. Final extension step of about 70° C. for about 5 minutes was also carried out. After enrichment PCR, libraries were purified with Beckman Coulter magnetic beads (Brea, Calif.) and about 80% ethanol wash, validated on Agilent bioanalyzer and DNA concentration was determined using Quant-iT PicoGreen dsDNA Reagent from Thermo Fisher Scientific (Eugene, Oreg.).

RNA-Seq Analysis:

Reads from two separate sequencing runs were concatenated to maximize sequencing depth and coverage. RNA-Seq data were analyzed using CLC Genomics Workbench v9.5 (QIAGEN Bioinformatics, Redwood City, Calif.). Paired-end reads were combined and reads were trimmed of any remaining adapter sequences using CLC's Illumina read import feature using default parameters. The Clostridum beijerinckii SA-1 genome (GenBank accession number CP006777) was downloaded from NCBI using the CLC GenBank browser. The SA1 nucleotide sequence was then converted into a genome track and the associated annotations were used to create a track for gene evidence. All reads were then mapped to the reference genome using the CLC RNA-Seq analysis feature with default parameters. Expression level data were reported as transcripts per million (TPM). Finally, differential expression analyses were performed using CLC's Advanced RNA-Seq plugin. The data generated in these analyses allow for the generation of volcano plots in OriginPro 2015 graphing software (OriginLab Corporation, Northhampton, Mass.). Bam files were deposited in NCBI (PRJNA390299).

Gas Calculations:

For gas solubility, Henry's law: C=k×p was used where: C is concentration, k is Henry's constant at 37° C. and p is partial pressure. The k values used were (in g/L): 0.0225 for CO; 1 for CO₂; 0.033 for O₂ and 0.0014 for H₂. To calculate gas consumption=(O−I+E)×(−1), where O is output, I is input, and E is the amount the cells endogenously generated under nitrogen. Positive values indicate consumption. Negative values indicate generation.

Stoichiometry:

For this calculation we used the methods previously reported⁵⁶.

Nitrite Pulse Experiments:

Continuous culture pulse experiments were performed with different concentrations of nitrite in the form of sodium nitrite (Sigma-Aldrich Inc., Saint Louis, Mo., USA). Exact conditions are detailed elsewhere herein.

### REFERENCES FOR EXAMPLE 1

1. Jang, Y. et al. Butanol production from renewable biomass: rediscovery of metabolic pathways and metabolic engineering. Biotechnol. J. 7, 186-198 (2012).
2. Xiong, W. et al. CO2-fixing one-carbon metabolism in a cellulose-degrading bacterium Clostridium thermocellum. PNAS 113, 13180-13185 (2016).
3. Wood, H. G. & Ljungdahl, L. G. in Variations in Autotrophic Life (eds. Shively, J. M. & Barton, L. L.) 201-250 (Academic Press, 1991).
4. Ljungdahl, L. G. The autotrophic pathway of acetate synthesis in acetogenic bacteria. Annu. Rev. Microbiol. 40, 415-450 (1986).
5. Köpke, M. et al. Clostridium ljungdahlii represents a microbial production platform based on syngas. Proc. Natl. Acad. Sci. 107, 15305-15305 (2010).
6. Dürre, P. Butanol formation from gaseous substrates. FEMS Microbiol Lett 363, 1-7 (2016).
7. Drake, H. L., Gofiner, A. S. & Daniel, S. L. Old acetogens, new light. Ann. N. Y. Acad. Sci. 1125, 100-128 (2008).
8. Qureshi N, Ezeji T C, Ebener J, Dien B S, Cotta M A, B. H. Butanol production by Clostridium beijerinckii Part I: use of acid and enzyme hydrolyzed corn fiber. Bioresour. Technol. 99, 5915-5922 (2008).
9. Qureshi, N., Saha, B. C. & Cotta, M. a. Butanol production from wheat straw by simultaneous saccharification and fermentation using Clostridium beijerinckii: Part II—Fed-batch fermentation. Biomass Bioenerg 32, 176-183 (2008).
10. Kumar, M. & Gayen, K. Developments in biobutanol production: New insights. Appl Energy 88, 1999-2012 (2011).
11. Branduardi, P., Branduardi, P. & Porro, D. n-butanol: challenges and solutions for shifting natural metabolic pathways into a viable microbial production. FEMS Microbiol. Lett. 263, 1-22 (2016).
12. Abrini, J., Naveau, H. & Nyns, E.-J. Clostridium autoethanogenum, sp. nov., an anaerobic bacterium that produces ethanol from carbon monoxide. Arch. Microbiol. 161, 345-351 (1994).
13. Younesi, H., Najafpour, G. & Mohamed, A. R. Ethanol and acetate production from synthesis gas via fermentation processes using anaerobic bacterium, Clostridium ljungdahlii. Biochem Eng J 27, 110-9 (2005).
14. Fernández-Naveira, Á., Abubackar, H. N., Veiga, M. C. M. C. & Kennes, C. Efficient butanol-ethanol (B-E) production from carbon monoxide fermentation by Clostridium carboxidivorans. Appl. Microbiol. Biotechnol. 100, 3361-3370 (2016).
15. Ezeji T C, Qureshi N, B. H., Ezeji, T. C., Qureshi, N. & Blaschek, H. P. Acetone butanol ethanol (ABE) production from concentrated substrate: reduction in substrate inhibition by fed-batch technique and product inhibition by gas stripping. Appl. Microbiol. Biotechnol. 63, 653-658 (2004).
16. Formanek, J., Mackie, R. & Blaschek, H. P. Enhanced butanol production by Clostridium beijerinckii BA101 grown in semidefined P2 medium containing 6 percent maltodextrin or glucose. Appl. Environ. Microbiol. 63, 2306-2310 (1997).
17. Papoutsakis, E. T. Equations and calculations for fermentations of butyric acid bacteria. Biotechnol. Bioeng. 26, 174-87 (1984).
18. Sandoval-Espinola, W. J. et al. Comparative phenotypic analysis and genome sequence of Clostridium beijerinckii SA-1, an offspring of NCIMB 8052. Microbiology 62, 2558-2570 (2013).
19. Monod, J. Recherches sur la croissance des cultures bacteriennes. Hermann Cie 80, (1942).
20. Chen, Y., Kim, J. K., Hirning, A. J., Josić, K. & Bennett, M. R. Emergent genetic oscillations in a synthetic microbial consortium. Science (80-.). 349, 986-989 (2015).
21. Heluane, H., Evans, M. R., Dagher, S. F. & Bruno-Bárcena, J. M. Meta-Analysis and functional validation of nutritional requirements of solventogenic Clostridia growing under butanol stress conditions and coutilization of D-glucose and D-xylose. Appl. Environ. Microbiol. 77, 4473-4485 (2011).
22. Jeon, B. Y., Jung, I. L. & Park, D. H. Conversion of carbon dioxide to metabolites by Clostridium acetobutylicum KCTC1037 cultivated with electrochemical reducing power. Adv. Microbiol. 2, 332-339 (2012).
23. Müller, V. Energy conservation in acetogenic bacteria. Appl. Environ. Microbiol. 69, 6345-6353 (2003).
24. Savage, M. D., Wu, Z., Daniel, S. L., Lundie, L. L. & Drake, H. L. Carbon monoxide dependent chemolithotrophic growth of Clostridium thermoautotrophicum. Appl. Environ. Microbiol. 53, 1902-1906 (1987).
25. Furdui, C. & Ragsdale, S. W. The role of pyruvate ferredoxin oxidoreductase in pyruvate synthesis during autotrophic growth by the Wood-Ljungdahl pathway. J. Biol. Chem. 275, 28494-28499 (2000).
26. Lin, Y. & Blaschek, H. P. Butanol production by a butanol-tolerant strain of Clostridium acetobutylicum in extruded corn broth. Appl. Environ. Microbiol. 45, 966-973 (1983).
27. Wang, Y., Li, X., Mao, Y. & Blaschek, H. P. Single-nucleotide resolution analysis of the transcriptome structure of Clostridium beijerinckii NCIMB 8052 using RNA-Seq. BMC Genomics 12,479 (2011).
28. Smith, K. S. & Ferry, J. G. Prokaryotic carbonic anhydrases. FEMS Microbiol Rev 24, 335-366 (2000).
29. Savage, M. D., Wu, Z., Daniel, S. L., Lundie, L. L. & Drake, H. L. Carbon monoxide-dependent chemolithotrophic growth of Clostridium thermoautotrophicum. Appl. Environ. Microbiol. 53, 1902-1906 (1987).
30. Tan, Y., Liu, J., Chen, X., Zheng, H. & Li, F. RNA-seq-based comparative transcriptome analysis of the syngas-utilizing bacterium Clostridium ljungdahlii DSM 13528 grown autotrophically and heterotrophically. Mol. Biosyst. 9, 2775-2784 (2013).
31. Pierce, E. et al. The complete genome sequence of Moorella thermoacetica (f. Clostridium thermoaceticum). Environ. Microbiol. 10, 2550-2573 (2008).
32. Diekert, G. B. & Thauer, R. K. Carbon monoxide oxidation by Clostridium thermoaceticum and Clostridium formicoaceticum. J. Bacteriol. 136, 597-606 (1978).
33. Papoutsakis, E. T. & Meyer, C. L. Equations and calculations of product yields and preferred pathways for butanediol and mixed-acid fermentations. Biotechnol. Bioeng. 27, 50-66 (1985).
34. Kim, B. H., Bellows, P., Datta, R. & Zeikus, J. G. Control of carbon and electron flow in Clostridium acetobutylicum fermentations: utilization of carbon monoxide to inhibit hydrogen production and to enhance butanol yields. Appl. Environ. Microbiol. 48, 764-770 (1984).
35. Drake, H., Kusel, K. & Matthies, C. in The Prokaryotes (eds. Eugene, R., Edward, F. D., Stephen, L., Erko, S. & Fabiano, T.) 545-577 (Springer Berlin Heidelberg, 2013). doi:10.1007/978-3-642-30194-0
36. Fröstl, J. M., Seifritz, C. & Drake, H. L. Effect of nitrate on the autotrophic metabolism of the acetogens Clostridium thermoautotrophicum and Clostridium thermoaceticum. J. Bacteriol. 178, 4597-4603 (1996).
37. Seifritz, C., Drake, H. L. & Daniel, S. L. Nitrite as an energy-conserving electron sink for the acetogenic bacterium Moorella thermoacetica. Curr. Microbiol. 46, 329-33 (2003).
38. Wohlfarth, G. & Diekert, G. Thermodynamics of methylenetetrahydrofolate reduction to methyltetrahydrofolate and its implications for the energy metabolism of homoacetogenic bacteria. Arch Microbiol 7, 378-381 (1991).
39. Schuchmann, K. & Müller, V. Autotrophy at the thermodynamic limit of life: a model for energy conservation in acetogenic bacteria. Nat. Rev. Microbiol. 12, 809-821 (2014).
40. Nataf, Y. et al. The Rnf complex of Clostridium ljungdahlii is a proton-translocating ferredoxin:NAD+ oxidoreductase essential for autotrophic growth. MBio 4, 1-8 (2013).
41. Stouthamer, A. H., Boogerd, F. C. & van Verseveld, H. W. The bioenergetics of denitrification. Antonie Van Leeuwenhoek 48, 545-553 (1983).
42. Cserzo, M., Wallin, E., Simon, I., von Heijne. & Elofsson, A. Prediction of transmembrane alpha-helices in prokaryotic membrane proteins: the dense alignment surface method. Prot. Eng. 10, 673-76 (1997).
43. Ragsdale, S. W. & Kumar, M. Nickel-containing carbon monoxide dehydrogenase/Acetyl-CoA synthase. Chem. Rev. 96, 2515-2540 (1996).
44. Lindahl, P. A. & Chang, B. The evolution of Acetyl-CoA synthase. Orig. Life Evol. Biosph. 31, 403-434 (2001).
45. Lindahl, P. a. Acetyl-coenzyme A synthase: the case for a Ni(p)(0)-based mechanism of catalysis. J. Biol. Inorg. Chem. 9, 516-524 (2004).
46. Whitham, J. M. et al. Characterization of Clostridium ljungdahlii OTA1: a non-autotrophic hyper ethanol-producing strain. Appl. Microbiol. Biotechnol. 100, DOI:10.1007/500253-016-7978-6 (2016).
47. Jones, S. W. et al. CO2 fixation by anaerobic non-photosynthetic mixotrophy for improved carbon conversion. Nat. Com. 7,12800 (2016).
48. Kobayashi, H. et al. Programmable cells: interfacing natural and engineered gene networks. Proc. Natl. Acad. Sci. 101, 8414-9 (2004).
49. Elowitz, M. B. & Leibler, S. A synthetic oscillatory network of transcriptional regulators. Nature 403, 335-338 (2000).
50. Sandoval-Espinola, W. J., Chinn, M. S. & Bruno-Barcena, J. M. Inoculum optimization of Clostridium beijerinckii for reproducible growth. FEMS Microbiol. Lett. 362, fnv164 (2015).
51. Thompson, J. D., Higgins, D. G. & Gibson, T. J. CLUSTAL W: improving the sensitivity of progressive multiple sequence alignment through sequence weighting, position-specific gap penalties and weight matrix choice. Nucleic Acids Res. 22, 4673-4680 (1994).
52. Merchant, N. et al. The iPlant Collaborative: cyberinfrastructure for enabling data to discovery for the life sciences. PLoS Biol. 14, e1002342 (2016).
53. Bolger, A. M., Lohse, M. & Usadel, B. Trimmomatic: a flexible trimmer for Illumina sequence data. Bioinformatics btu170 (2014). doi:10.1093/bioinformatics/btu170
54. Kim, D. et al. TopHat2: accurate alignment of transcriptomes in the presence of insertions, deletions and gene fusions. Genome Biol. 14, R36 (2013).
55. Trapnell, C. et al. Differential analysis of gene regulation at transcript resolution with RNA-seq. Nat. Biotechnol. 31, 46-53 (2013).
56. Erickson, L. E., Selga, S. E. & Viesturs, U. E. Application of mass and energy balance regularities to product formation. Biotech. Bioeng. 20, 1623-1638 (1978).

1. A system comprising:
a fermentation vessel, wherein the fermentation vessel is configured to receive an inorganic carbon source; and
a culture of solventogenic Clostridium beijerinckii (C. beijerinckii), wherein the culture of solventogenic C. beijerinckii is contained within the fermentation vessel.

2. The system of claim 1, wherein the culture of solventogenic C. beijerinckii is a mixotrophic culture of solventogenic Clostridium beijerinckii.

3. The system of claim 1, wherein the culture of solventogenic C. beijerinckii is a high density culture of solventogenic C. beijerinckii.

4. The system of claim 1, wherein the inorganic carbon source is a greenhouse gas.

5. The system of claim 1, wherein the inorganic carbon and electron source is syngas.

6. The system of claim 1, wherein the carbon source is CO₂ and electron source is H₂.

7. The system of claim 1, wherein the inorganic carbon and electron source is up to 20% (v/v) CO.

8. The system of claim 1, wherein the inorganic carbon source is up to 20% (v/v) CO₂.

9. The system of claim 1, wherein the electron source is up to 8% (v/v) H₂.

10. A method of fixing inorganic carbon, the method comprising:
fermenting a carbon source using a culture of solventogenic Clostridium beijerinckii (C. beijerinckii).

11. The method of claim 10, wherein the culture of solventogenic C. beijerinckii is a mixotrophic culture of solventogenic C. beijerinckii.

12. The method of claim 10, wherein the culture of solventogenic C. beijerinckii is a high density culture of solventogenic C. beijerinckii.

13. The method of claim 10, wherein the inorganic carbon and electron source is a greenhouse gas.

14. The method of claim 10, wherein the carbon and electron source is syngas.

15. The method of claim 10, wherein the carbon and electron source is up to 20% (v/v) CO.

16. The method of claim 10, wherein the carbon source is up to 20% (v/v) CO₂.

17. The method of claim 10, wherein the electron source is up to 8% (v/v) H₂.

18. The method of claim 10, wherein the carbon source is at least 5% (v/v) CO₂ and electron source 2.5% (v/v) H₂.

19. The method of claim 10, wherein the step of fermentation is carried out at about 37° C.

20. The method of claim 10, wherein the carbon source is syngas and wherein the syngas is about 9%, about 32%, about 63%, or 100%.
